(19) United States
(12) Patent Application Publication
(10) Pub. No.: US 2009/0275642 A1
(43) Pub. Date: Nov. 5, 2009

(54) DNA COMPOSITION FOR ELICITING AN IMMUNE RESPONSE AGAINST TUMOR-ASSOCIATED MACROPHAGES

(76) Inventors: Ralph A. Reisfeld, La Jolla, CA (US); Rong Xiang, San Diego, CA (US); Yunping Luo, San Diego, CA (US)

Correspondence Address: Olson & Cepuritis, LTD., 20 NORTH WACKER DRIVE, 36TH FLOOR, CHICAGO, IL, 60606, US

(21) Appl. No.: 12/311,379

(22) PCT Filed: Oct. 5, 2007
(86) PCT No.: PCT/US07/21414

§ 371 Date: Mar. 26, 2009

Related U.S. Application Data
(60) Provisional application No. 60/849,927, filed on Oct. 6, 2006.

(51) Int. Cl. A61K 48/00; C07H 21/04; C12N 15/63; A61P 35/00

(52) U.S. Cl. 514/44 R; 536/23.5; 435/320.1

(57) ABSTRACT

The present invention provides a DNA composition comprising a DNA minigene construct that encodes for a polypeptide comprising a plurality of immunogenic fragments of a cysteine endopeptidase that is expressed in tumor-associated cells. The immunogenic fragments are joined together serially by a linker peptide between each successive fragment in the polypeptide. The polypeptide is capable of eliciting an immune response against the tumor-associated cells, is expressible in immune cells, and is incorporated in a pharmaceutically acceptable carrier.

### CROSS-REFERENCE TO RELATED APPLICATIONS

This application claims the benefit of U.S. Provisional Application Ser. No. 60/849,927, filed on Oct. 6, 2006, which is incorporated herein by reference.

### GOVERNMENTAL RIGHTS

This invention was made with United States government support under Grant Nos. DAMD17-02-0137 and DAMD17-02-0562 from the Department of Defense, as well as Grant Nos. W81XWH-05-1-0091 and W81XWH-05-1-0318 from the Congressionally Directed Medical Research Program. The government has certain rights in this invention.

### FIELD OF THE INVENTION

This invention relates to deoxyribonucleic acid (DNA) compositions encoding suitable molecules effective for eliciting an immune response against tumor-associated macrophages. More particularly this invention relates to DNA compositions encoding at least one epitope of an endopeptidase, such as legumain, which is expressed in a tumor-associated cell, such as a tumor-associated macrophage. This invention also relates to methods of using the DNA composition to inhibit tumor growth and tumor metastases.

### BACKGROUND OF THE INVENTION

Tumor-associated macrophages (TAMs) are associated with tumor progression and metastasis. A novel anti-tumor strategy is to immunize against molecules overexpressed by TAMs and thereby remodel the tumor microenvironment, which attracts these macrophages and mediates their function. (see Oosterling et al. 2005 J. Pathol. 207:147-155; Emens et al. 2005 Endocr. Relat Cancer 12:1-17). TAMs consist primarily of a polarized M2 (CD206⁺, F4/80⁺) macrophage population with little cytotoxicity for tumor cells because of their limited production of nitric oxide and proinflammatory cytokines (see Mills et al. 2000 J. Immunol. 164:6166-6173).

TAMs also possess poor antigen presenting capability and effectively suppress T cell activation. In fact, these macrophages of M2 phenotype actually promote tumor cell proliferation and metastasis by secreting a wide range of growth and pro-angiogenesis factors as well as metalloproteinases, and by their involvement in signaling circuits that regulate the function of fibroblasts in the tumor stroma (Mantovani et al. 2004, Novartis. Found. Symp. 256:137-145).

Currently, anti-TAM effects induced by small molecule inhibitors reportedly have contributed to tumor suppression (see Lewis et al. 2005 Am. J. Pathol. 167:627-635; and Mantovani et al. 2004, Eur. J. Cancer 40:1660-1667). For example, yondelis, an antineoplastic agent, has a selective cytotoxic effect on TAMs, thereby significantly reducing their production of IL6 and CCL2, which contribute to growth suppression of inflammation-associated human tumors (see Allavena et al. 2005, Cancer Res. 65:2964-2971). Another such example is provided by a biphosphonate compound, zoledronic acid, which suppresses MMP9 secretion by TAMs, thereby inhibiting tumor metalloproteinase activity and diminishing the association of VEGF with its tyrosine kinase receptors on proliferating endothelial cells (see Giraudo et al. 2004, J. Clin. Invest. 114:623-633).

In a different experimental model, the chemokine CCL5 was shown to be important in the recruitment of TAMs. An antagonist of this chemokine reduced the tumor infiltrate and slowed tumor growth (see Robinson et al. 2003, Cancer Res. 63:8360-8365). Hence, although the therapeutic targeting of TAMs is still a relatively new approach, initial clinical results are encouraging, as they suggest that targeting TAMs may complement more conventional cancer treatment regimens.

Legumain is an entirely novel evolutionary offshoot of the C13 family of cysteine proteases (see Ishii 1994, Methods Enzymol. 244:604-615), and is well conserved in plants and mammals, including humans. Legumain was first identified in plants as a processing enzyme of storage proteins during seed germination, and was subsequently identified in parasites and then in mammals. Legumain is a robust acidic cysteine endopeptidase with remarkably restricted specificity absolutely requiring an asparagine at the P1 site of its substrate sequence (see Chen et al. 1997, J. Biol. Chem. 272:8090-8098).

In the present application, the selection of legumain as a target for tumor therapy is based on the fact that the gene encoding this endopeptidase was found to be highly up-regulated in many murine and human tumor tissues, but absent or only present at very low levels in all normal tissues from which such tumors arise. Importantly, overexpression of legumain occurs under such stress conditions as tumor hypoxia, which leads to increased tumor progression, angiogenesis and metastasis.

Legumain is a particularly preferred target endopeptidase for the compositions and methods of the present invention, due to the observation that legumain is heavily overexpressed by TAMs in murine breast tumor tissues, as confirmed by gene expression profiling and immunohistochemistry. TAMs have a particularly abundant expression of legumain in the tumor stroma. In contrast, classical macrophages of M1 phenotype, which perform important immune-surveillance and antigen presentation functions, do not express legumain. Consequently, targeting TAMs that overexpress legumain does not interfere with the biological functions of (M1) macrophages, including cytotoxicity and antigen presentation. The present invention provides DNA compositions to induce an immune response against TAMs that overexpress legumain or other endopetidases that are overexpressed in TAMs, and which are useful for treatment of tumors and tumor metastases.

### SUMMARY OF THE INVENTION

A DNA composition of the present invention comprises a DNA construct that encodes a polypeptide comprising at least an immunogenic fragment of a cysteine endopeptidase that is overexpressed in tumor-associated cells (e.g., tumor-associated macrophages). The DNA construct includes structural elements that facilitate the expression of the polypeptide in immune cells of a subject to which the DNA construct has been administered. Preferably, the endopeptidase comprises legumain or at least one immunogenic fragment (e.g., epitope) thereof. The DNA construct is incorporated in a pharmaceutically acceptable carrier so that it can be administered to a patient. The composition can encode a single immunogenic fragment of the endopeptidase (e.g., an epitope of legumain), a polypeptide comprising two or more immunogenic fragments of the endopeptidase (i.e., an immunogenic polypeptide), an entire endopeptidase protein, or any portion thereof that will elicit an immune response in the subject. Preferably, the DNA construct encodes for human legumain having the amino acid residue sequence consisting of SEQ ID NO: 2, a protein that has at least 80% sequence identity with SEQ ID NO: 2 (e.g., human legumain, porcine legumain, or mouse legumain), or an immunogenic fragment thereof, and which is expressible in immune cells of a subject to which the DNA construct is administered. The DNA constructs of the invention are useful for inhibiting tumor growth and tumor metastases.

The DNA construct can be a naked DNA construct, preferably in the form of a plasmid. Such naked DNA constructs can be incorporated in a liposome delivery vehicle, a polymeric delivery vehicle, or administered by electroporation, a gene gun, and the like, if desired. In some preferred embodiments the DNA construct is incorporated in an attenuated viral vector or an attenuated bacterial vector.

In a preferred embodiment, the DNA construct is incorporated in an attenuated bacterial vector, such as an attenuated Salmonella typhimurium, e.g., the doubly attenuated (AroA⁻, dam⁻) strain of Salmonella typhimurium.

Optionally, the DNA composition can also comprise a DNA construct encoding an immune effector protein, such as a cytokine. Preferred cytokines include CCL21, IL-2, and CD40LT.

In a particularly preferred embodiment, the DNA composition of the invention comprises a DNA minigene construct that encodes for an immunogenic polypeptide comprising a plurality of immunogenic fragments (e.g., epitopes) of a cysteine endopeptidase (e.g., legumain) that is highly expressed in tumor-associated cells. The immunogenic polypeptide is capable of eliciting an immune response against the tumor-associated cells, is expressible in immune cells, and is incorporated in a pharmaceutically acceptable carrier. The immunogenic fragments are joined together serially by a linker peptide between each successive fragment in the polypeptide. The linker peptides typically are at least three amino acid residues in length and preferably comprise the amino acid sequence AAA or AAY. As used herein, the term “linker peptide” refers to a sequence of at least two amino acid residues, preferably at least three amino acid residues, which form an amino acid residue sequence that differs from the natural endopeptidase when linked together with the immunogenic fragments of the endopeptidase. Typically the combination of linker peptides and immunogenic fragments of the endopeptidase will comprise a polypeptide of less than about 100 amino acid residues in length, more preferably about 19 to about 62 amino acids in length (e.g., two to about five immunogenic fragments of 8 to 10 amino acids each, joined together by one to four linker peptides of three amino acids each). Preferably, the DNA minigene construct encodes for immunogenic fragments of human legumain (SEQ ID NO: 2).

The DNA compositions of the present invention can act as vaccines that target tumor-associated macrophages that express a cysteine endopeptidase, such as legumain, providing a highly selective target for T cell-mediated cancer immunotherapy. The approach of targeting an endopeptidase such as legumain, which is expressed by tumor-associated macrophages has several advantages over therapies directed against antigens that are solely expressed by tumor cells themselves. For example, legumain is highly overexpressed in TAMs, and is thus not impaired by down regulated MHC-antigen expression, as is frequently the case in tumor cells. In addition, tumor cells often become increasingly resistant to T cell mediated killing due to defects in apoptosis signaling pathways, upregulation of antiapoptotic proteins, or immunosuppressive effects on cytotoxic T lymphocytes (CTLs). Targeting TAMs that express legumain allows for a therapeutic composition to treat a number of different malignancies, in contrast to therapies involving antigens that are expressed solely by specific tumor types.

In one preferred embodiment, the DNA compositions of the present invention break peripheral T cell tolerance against the legumain self-antigen by delivering its cDNA of DNA encoding one or more immunogenic fragments thereof, as an oral DNA composition with an attenuated bacterial delivery vector (e.g., an attenuated strain of Salmonella typhimurium). In such embodiments, the DNA composition is contacted with antigen presenting cells (APCs) in a secondary lymphoid organ, i.e., the Peyer's patches of the small intestine. In a prophylactic approach, the T cell-mediated antitumor immune response induced by vaccination with a DNA composition of the invention inhibited tumor growth in multiple murine tumor models. The present DNA compositions also significantly suppress the dissemination of established pulmonary metastases in a therapeutic model of CT26 colon carcinoma.

A preferred DNA composition comprises an attenuated Salmonella carrier, such as a doubly attenuated strain of S. typhimurium, e.g., the strain designated as RE 88, which includes the dam⁻ and AroA⁻ mutations, and is available from Remedyne Corporation (Goleta, Calif.). In the present invention, the attenuated Salmonella carrier is transfected so that is includes the DNA construct encoding the endopeptidase (e.g., legumain) or a polypeptide encoding an immunogenic fragment thereof. The endopeptidase or polypeptide is expressible in immune cells of a mammal to which it is administered. The bacteria do not themselves express the legumain or polypeptide, but rather deliver the DNA to immune cells, such as macrophages and dendritic cells (DCs), which in turn express endopeptidase or the polypeptide comprising the immunogenic fragment thereof. Such compositions can provide prolonged antitumor effects in murine models. Furthermore, in vivo depletion experiments of T cells indicated the involvement of CD8⁺ but not CD4⁺ T cells in the immunogenic response associated with compositions encoding legumain and encoding polypeptides comprising immunogenic fragments of legumain. The observed cytotoxic effect mediated by CD8⁺ T cells in vitro was specifically directed against target TAMs that overexpress the legumain antigen.

The DNA compositions of the present invention also can incorporate DNA constructs that encode immune effector molecules as adjuvants for the composition. Such immune effector molecules include, for example, IL-2, an inducer of T cell proliferation, CCL21, a chemokine that chemo-attracts mature dendritic cells, and naive T cells, as well as CD40LT, a known inducer of dendritic cell maturation. The nucleic acids encoding the immune effector protein preferably are incorporated into a plasmid. The legumain and immune effector protein DNA constructs can be incorporated into the same plasmid or into two separate plasmids. The CTL-response induced against TAMs can inhibit the growth of a variety of tumors, and is not specific to a particular tumor type.

The present invention also provides a method of inhibiting tumor growth and tumor metastases in a mammal comprising administering to a mammal a DNA composition of the invention in an amount sufficient to elicit an immune response against TAMs that express legumain.

Another aspect of the present invention is an effective combination therapeutic regimen that combines chemotherapy and treatment with a DNA composition of the invention. In this method embodiment of the present invention various chemotherapeutic agents, such as doxorubicin, paclitaxel, and/or cyclophosphamide, which do not cause bone marrow suppression when administered at the maximum tolerable dose (MTD), are administered to the patient in conjunction with a DNA composition of the invention comprising a DNA construct that encodes a TAM-expressed endopeptidase, such as legumain or a polypeptide encoding an immunogenic portion thereof, preferably comprising a minigene construct that comprises at least two immunogenic fragments of the endopeptidase jointed together serially by a linker peptide between each successive immunogenic fragment in the polypeptide.

Another preferred method embodiment is a method of inhibiting tumor growth or tumor metastases in a mammal (e.g., a human) comprising the steps of administering to a mammal a DNA composition of the invention in an amount sufficient to elicit an immune response against TAMs in the mammal, which overexpress an endopeptidase such as legumain, and subsequently administering to the mammal an antitumor effective amount of an antitumor chemotherapeutic agent.

Preferably, the mammal treated by the methods of the present invention is a human.

In the method embodiments of the present invention, the DNA compositions can be administered enterally, such as by oral administration, or parenterally, such as by injection or intravenous infusion. Preferably, the compositions are administered orally. The compositions can be packaged in sealed containers and labeled with information useful for a clinician to effectively administer the composition.

The DNA compositions of the present invention are useful for treatment and prevention of a number of disease states. For example, a patient suffering from colorectal cancer, breast cancer, lung cancer, and the like, can benefit from immunization by the compositions of the present invention. The compositions of the present invention are also useful for investigating the role of legumain in various forms of cancer.

### BRIEF DESCRIPTION OF THE DRAWINGS

FIG. 1. Legumain is highly expressed on tumor-associated macrophages in the tumor stroma. (A) Legumain expression on TAMs was clearly evident as shown in Panel A. Tumor-infiltrating macrophages were visualized by H/E staining as indicated by arrows. Legumain expression is indicated by double staining with anti-legumain Ab combined with anti-CD68 Ab. (Magnification ×35) (B) Increased legumain expression on TAMs was confirmed by flow cytometric analyses with double positive populations of CD206⁺/F4/80⁺ M2 macrophages that were isolated from fresh tumor tissue. (C) Multiple color flow cytometry demonstrated up-regulation of the M2 macrophage marker CD206 on RAW cells after being cultured with IL-4, IL-10 and IL-13 (10 ng/ml). Legumain was shown to be highly expressed on F4/80⁺/CD206⁺ positive RAW cells cultured with IL-4, IL-10 and IL-13 as indicated in the upper photo when compared with wild type RAW cells depicted in the lower photo. (D) Confirmation of legumain expression on RAW cells by Western blotting following stimulation with IL-4, IL-13 and IL-10, either singularly or combined.

FIG. 2. Targeting of legumain expressing cells results in suppression of tumor progression. (A) Schematic of a DNA composition of the invention constructed with the pCMV/myc/cyto vector backbone where the legumain gene was fused to the C-terminus of mutant polyubiquitin. The entire fragment was inserted and protein expression was demonstrated by Western blotting. (B) Prophylactic model: the vaccination schedule was designed for three immunizations at 1-week intervals followed by an i.v. challenge with about 2×10⁵ D121 non-small cell lung cancer cells, about 5×10⁴ CT26 colon cancer cells, and mammary fat pad injection with about 7×10³ 4T1 breast cancer cells. Lung weights were determined 24 days (D121 or CT-26) or 30 days (4T1) after tumor cell challenge and analyzed in each group. Differences between the two control groups (PBS and/or empty vector) and the treatment group were statistically significant **P<0.005. Normal lung weight=0.2 g. (C) Therapeutic model: groups of BALB/c mice (n=8) were initially injected into the mammary fat pad with about 7×10³ 4T1 breast cancer cells and thereafter vaccinated three times on days 3, 7 and 11 with PBS, empty vector or the pLegumain DNA composition, respectively, and primary tumors excised on day 12. Survival plots represent results for 8 mice in each of the treatment and control groups. Difference between the empty vector control group and the treatment group was statistically significant **P<0.005.

FIG. 3. TAM population in the tumor stroma is decreased by CD8+ specific CTLs induced by the legumain-based DNA composition. (A) RAW macrophage cells highly express legumain after culturing with 10 ng/ml IL-4, IL-10, IL-13 and serve as target cells in a 4-hour ⁵¹Cr-release assay. Splenocytes isolated from mice immunized with the pLegumain composition effectively killed RAW cells treated with these cytokines in vitro at different effector-to-target cell ratios, but failed to induce cytotoxic killing of unstimulated RAW cells lacking legumain expression. **P<0.005 compared to control groups. (B) Flow cytometry detects the percentage of TAM populations with specific macrophage markers (CD206 and F4/80) in tumor tissue after vaccination. The percentage of TAM populations among tumor tissue cells, isolated from mice treated with the DNA composition of the invention was shown to be reduced. There was no decrease in TAM populations isolated from mice treated with either empty vector or pLegumain following CD8⁺ T cell depletion (**P<0.005). (C) The results of flow cytometry were confirmed by immunohistochemical staining evaluated by confocal microscopy. The population of TAMs in the tumor stroma was dramatically decreased after vaccination. Magnification ×5 (H/E), ×35 (Control, Empty Vector and pLegumain).

FIG. 4. MHC-class I antigen restricted specific CD8⁺ T cell response against legumain expressing cells. (A) FACS plots show that DNA treatment enhances expression of co-stimulatory molecules by DCs. Lymphocytes from Peyer's patches obtained 3 days after vaccination were stained with FITC labeled anti-CD11cAb in combination with PE conjugated anti-CD80 Ab, anti-MHC class I or anti-CD40 Abs. (*P<0.05, compared to control groups). (B) Intracytoplasmic INF-gamma release of CD8⁺ T cells was measured by FACS analysis. **P<0.005, compared to control groups. (C) Production of specific INF-gamma was verified at the single cell level by ELISPOT. This is indicated for lymphocytes from immunized mice restimulated with either legumain⁺ 4T1 tumor tissue cells or legumain⁻ 4T1 cells, as depicted by the number of immunospots formed per well. **P<0.005 compared to treatment group without stimulation. ##P<0.005 compared to control groups. (D) Splenocytes isolated from treated mice were effective in killing TAMs by using a ⁵¹Cr release assay (*P<0.01, compared to control groups). Inhibition experiments with Abs against H2Kd/H-2Dd MHC class I antigens showed that T cell mediated tumor cell lysis was MHC class I Ag restricted. In vivo depletions of CD4⁺ or CD8⁺ T cells indicated that lymphocytes isolated from vaccinated mice, which were thereafter depleted of CD8⁺ T cells, failed to induce cytotoxic killing of target cells, Depletion of CD4⁺ T cells did not abrogate cytotoxic killing of these same target cells. *P<0.01 compared to PBS or empty vector group.

FIG. 5. Abrogation of TAMs results in decreases of growth factors release, tumor cell migration and metastases. (A) The DNA composition of the invention decreased the release of growth factors by TAMs. 4T1 breast tumor tissue and mouse serum were harvested 12 days after vaccinations and tumor cell challenge. After 24 hours or 48 hours culturing, the supernatants of tumor tissue cells were harvested, and the concentrations of TGF-beta, TNF-alpha and VEGF in serum or supernatants measured by ELISA. There were significant differences between the treatment group and control groups. *P<0.01, **P<0.005. (B) Immunohistochemical staining was performed to determine expression of these growth factors in the tumor microenvironment. The vaccine treatment groups showed that VEGF, TGF-beta and MMP-9 releases were decreased after a reduction in TAMs, compared with the empty vector groups. (C) A transwell migration assay was performed to determine tumor cell migration after vaccination. The number of migrating cells was markedly reduced after vaccination. ***P<0.001 compared to the empty vector group. (D) In vivo experiments were performed to determine the ability of mice to form tumor metastases. The mice were treated with the vaccine within the therapeutic setting as described above. Tumor metastasis scores and lung weights were measured 25 days after primary tumor excision. The metastasis scores are expressed as the % lung surface covered by fused metastatic foci: 0=none; 1=<5%, 2=5% to 50%, and 3=>50%. Differences in lung weights between the group of mice treated with vaccine and all control groups were statistically significant (**P<0.005).

FIG. 6. Elimination of TAMs results in a reduction of tumor angiogenesis. Suppression of VEGF-induced angiogenesis: BALB/c mice were vaccinated with S. typhimurium transfected with either empty vector, pLegumain, or pLegumain after either CD8⁺ or CD4⁺ T cell depletion in vivo, respectively. One week after the last vaccination, Matrigel was implanted s.c. into the midline of the abdomen of mice. Vascularization was induced by VEGF or bFGF. (A) The images were taken by a digital camera 6 days after Matrigel plug implantation. Additionally, the section of Matrigel plugs stained with Massion's trichrome indicate blood vessel growth in Matrigel plugs as highlighted by arrows (Magnification ×5). (B) Quantification of vessel growth was performed after in vivo staining of endothelium with FITC-labeled isolectin B4 and evaluation by fluorimetry. There was a decrease in the VEGF-induced neovasculature only after vaccination with the vector encoding legumain but not after vaccination with the empty vector or with pLegumain after depletion of CD8⁺ T cells. **P<0.005, *P<0.01 compared to the legumain treatment group. (C) Immunohistochemical staining was performed and evaluated by confocal microscopy. The cross sections of matrigel plugs were stained to determine the cell type that grew in or migrated into these plugs. The images indicate that endothelial cells with the CD31 marker or macrophages with the CD68 marker grew in or migrated into Matrigel plugs as indicated by arrows (Magnification ×35). H/E staining served as a control (Magnification ×5).

FIG. 7. The 4T1 cell line was stably transfected by a retrovirus harboring the legumain plasmid and then used as target cells for splenocytes from immunized mice (Panel A, left photos have a 5× magnification; right photos have a 35× magnification), the images were taken 2 days after transfection and the positive cells are indicated by arrows. The ⁵¹Cr release assay data are shown in Panel B. The splenocytes isolated from mice immunized with the pLegumain DNA composition were effective in killing 4T1 cells transfected with legumain (*P<0.01, compared to empty vector control groups). The tumor specific T cell-mediated killing was specific for legumain since normal 4T1 cells, lacking in legumain expression, were not lysed.

FIG. 8 shows the nucleotide sequence of a nucleic acid encoding human legumain (SEQ ID NO: 1).

FIG. 9 shows the amino acid residue sequence (SEQ ID NO: 2) of human legumain.

FIG. 10 shows the nucleotide sequence (SEQ ID NO: 3) of a nucleic acid encoding murine legumain.

FIG. 11 shows the amino acid residue sequence (SEQ ID NO: 4) of murine legumain.

FIG. 12 shows the nucleotide sequence (SEQ ID NO: 5) of a nucleic acid encoding human IL-2.

FIG. 13 shows the amino acid residue sequence of human IL-2 (SEQ ID NO: 6).

FIG. 14 shows the nucleotide sequence (SEQ ID NO: 7) of a nucleic acid encoding human CCL21.

FIG. 15 shows the amino acid residue sequence of human CCL21 (SEQ ID NO: 8).

FIG. 16 shows the nucleotide sequence (SEQ ID NO: 9) of a nucleic acid encoding human CD40L.

FIG. 17 shows the amino acid residue sequence of human CD40L (SEQ ID NO: 10).

FIG. 18 shows the nucleotide sequence of ubiquinated murine legumain (SEQ ID NO: 11).

FIG. 19 shows the amino acid residue sequence of ubiquinated murine legumain (SEQ ID NO: 12).

FIG. 20 shows the amino acid residue sequence of murine legumain epitope sequences.

FIG. 21 shows a schematic representation of plasmids encoding legumain minigenes comprising immunogenic fragments of legumain.

FIG. 22 demonstrates that the pCMV-Kb/Kd minigene composition protects mice from challenge with D2F2 breast carcinoma cells. Groups of BALB/c mice (n=8) were immunized 3 times at 1-week intervals with doubly attenuated Salmonella typhimurium RE-88 harboring the vectors indicated. Mice were challenged 1 week after the last immunization by i.v., injection of 2×10⁵ D2F2 breast carcinoma cells. A. Schematic of experimental protocol. B. Mean tumor volume of mice 5 to 25 days after tumor cell challenge. C. Tumor weight of mice 25 days after cell challenge, *, P<0.05 compared to empty vector control group.

FIG. 23 demonstrates that the pKd minigene vaccine prevents D2F2 breast carcinoma metastasis in syngeneic BALB/c mice. Groups of mice (n=8) were immunized 3 times at 1-week intervals by gavage with attenuated Salmonella typhimurium RE-88 harboring the vectors indicated. Mice were challenged 2 weeks after the last immunization by i.v., injection of 1×10⁵ D2F2 breast carcinoma cells. A. Schematic of experimental protocol. B. Average lung metastasis score of mice from each experimental group 25 days after tumor cell challenge. Tumor metastasis scores on lungs were established by estimating the % of surface area covered by fused metastases as follows: 0, no metastases; 1, <20%; 2, 20% to 50% and 3, >50% represented by individual symbols for each treatment group. * P<0.05 compared to empty vector control group.

FIG. 24 illustrates IFN-γ release in legumain-specific T cells induced by the pCMV-Kb/Kd minigene composition. A. The expression of legumain by 4T1 cells freshly harvested from tumor tissues were used as stimulator cells. Flow cytometry was performed to indicate the extent of legumain expression on those cells. B. Production of IFN-γ was verified at the single cell level by ELISPOT as lymphocytes from immunized mice were restimulated with either legumain⁺ 4T1 tumor cells freshly harvested from tumor tissue or legumain⁻ 4T1 tissue culture cells. IFN-γ release is indicated by the number of immunospots formed per well.

P<0.05, compared with groups of mice whose lymphocytes were not stimulated by legumain⁺ tumor cells. #P<0.05, compared with control groups.

FIG. 25 illustrates the specific CTL killing of legumain positive macrophage cells induced by the pCMV-Kb/Kd minigene composition. A. The expression of legumain by the macrophage cell line RAW is indicated after culture with IL-4, IL-10 and IL-13. Western blot analysis was performed to indicate legumain expression on those cells. B. Groups of immunized BALB/c mice (n=4) were sacrificed 2 weeks after the last immunization and splenocytes isolated from them were stimulated with irradiated 4T1 legumain⁺ cells for 5 days. Thereafter, cytotoxicity assays were performed with either wild type RAW legumain⁻ cells (lower panel), or RAW legumain⁺ cells as target cells (upper panel). * P<0.05, compared to empty vector control group while using RAW legumain⁺ cells as target cells.

FIG. 26 illustrates suppression of angiogenesis in syngeneic BALB/c mice induced by the pCMV-Kb/Kd minigene composition. A. Result of Matrigel assay. Matrigel was implanted into mice vaccinated with either empty vector, pCMV-Db/Dd or pCMV-Kb/Kd vaccines. The measurement of hemoglobin (Hb) concentration in Matrigel plugs was performed for quantification of blood vessel growth. The average Hb concentration of Matrigel plugs from each group of mice is depicted by the bar graph (n=4; mean±SD). *, P<0.05, compared to empty vector control group. B. Masson's trichrome staining of Matrigel sections prepared 7 days after Matrigel plug implantation. Arrows indicate blood vessels in the Matrigel plug.

### DETAILED DESCRIPTION OF PREFERRED EMBODIMENTS

The present invention provides a DNA composition, which targets the tumor-associated cells such as tumor-associated macrophages (TAMs). The DNA composition comprises a DNA construct that encodes a cysteine endopeptidase that is overexpressed in tumor-associated cells, or at least one immunogenic fragment thereof that is capable of eliciting an immune response against the tumor-associated cells, incorporated in a pharmaceutically acceptable carrier. Preferably, a DNA composition comprises a DNA minigene construct that encodes for an immunogenic polypeptide comprising a plurality of immunogenic fragments of a cysteine endopeptidase (e.g., legumain) that is highly expressed in tumor-associated cells, wherein the polypeptide is capable of eliciting an immune response against the tumor-associated cells, and is expressible in immune cells. The minigene embodiment, the plurality of immunogenic fragments of the endopeptidase are joined together serially by a linker peptide between each successive fragment in the polypeptide.

Preferably, the cysteine endopeptidase is legumain (e.g., human legumain; SEQ ID NO): 2). The DNA construct can encode a single immunogenic fragment of the cysteine endopeptidase (e.g., an epitope), but preferably encodes a polypeptide comprising two or more immunogenic fragments of the endopeptidase.

The term “DNA construct” as used herein and in the appended claims refers to a DNA structure that encodes for a protein or polypeptide of interest, such as legumain or an immunogenic legumain fragment (epitope), collectively referred to as “legumain DNA”, as well as proteins, such as IL-2, CCL21, CD40L and the like. Preferably, each immunogenic fragment comprises about 8 to 10 amino acid residues in length. DNA constructs include any DNA that can be transcribed in target cells, including linear DNA and plasmid DNA, as well as DNA which has been incorporated into the genetic material of a cell or virus. Preferably, the DNA construct is a DNA that has been incorporated in a viral or bacterial delivery vector, e.g., an attenuated viral or bacterial vector that is non-pathogenic. When a subject is treated with a composition of the invention, the legumain DNA is delivered to immune cells (e.g., macrophages and dendritic cells), which then express the protein or polypeptide including an immunogenic fragment thereof. Viral and bacterial carriers of the legumain DNA do not themselves express legumain.

In some preferred embodiments, the DNA construct is a minigene construct that encodes an immunogenic polypeptide comprising a plurality (e.g., 2 to 5) of immunogenic fragments of an endopeptidase that is highly expressed in TAMs (e.g., legumain). As used herein, the term “minigene” refers to DNA constructs that encode multiple portions (fragments) of a protein of interest, which are linked together, preferably by small peptides of at least three amino acids. Thus minigenes encode polypeptides that include immunogenic portions of the protein of interest, but do not encode the entire protein of interest. Preferably, a minigene construct encodes a polypeptide that comprises about 2 to 5 immunogenic fragments (i.e., peptide sequences from epitope regions of the protein), preferably joined together by linking peptides. The minigene can also include sequences that encode leader sequences and/or other sequences that are useful for facilitating expression or transport of the polypeptide.

As noted above, the immunogenic fragments in the polypeptide preferably are joined together by a linker or spacer peptide between each fragment. The linker peptide preferably comprises at least three amino acid residues (e.g., AAA or AAY). Preferably, the DNA construct encoding an immunogenic fragment of legumain also encodes a leader sequence, such as an endoplasmic reticulum (ER) leader sequence, connected to the N-terminus of the polypeptide encoded by the DNA construct by a linker peptide. When the DNA construct encodes a polypeptide comprising two or more immunogenic legumain fragments, the leader sequence preferably is linked to the first immunogenic fragment from the N-terminus of thereof. Preferably, immunogenic fragments of the endopeptidase consist of about 8 to 10 contiguous amino acid residues from one or more epitope region thereof.

Immunogenic fragments of endopeptidases such as legumain, including human legumain, can be identified by methods well known in the art, such as the HLA Binding Predictions program provided by the Bioinformatics & Molecular Analysis Section (BIMAS) of the National Institutes of Health (NIH) at the NIH www website, which is incorporated herein by reference.

The DNA compositions of the present invention stimulate formation of CTLs that are active against tumor-associated macrophages that express the endopeptidase. Such tumor-associated macrophages are selectively targeted by CTLs that are produced in response to immunization by the DNA compositions of the invention. Elimination or abrogation of TAMs changes the tumor microenvironment, resulting in suppression of tumor growth and tumor metastases.

As used herein, the term “immunity” refers to long term immunological protection against the virulent form of the infectious agent or tumor antigen. The term “immunization” refers to exposure to an antigen of a pathogenic agent derived from a non-virulent source, which results in immunity to the pathogen in the treated subject.

A DNA construct useful in a DNA composition of the present invention preferably comprises a nucleic acid that encodes a polypeptide comprising legumain (e.g., human legumain) or immunogenic fragments of legumain, and which is operably linked to regulatory elements needed for gene expression in immune cells. In a preferred embodiment, the DNA construct encodes for full length human legumain protein (SEQ ID NO: 2) or a polypeptide sharing a high degree of homology of at least about 80% therewith (e.g., porcine legumain, mouse legumain, ant the like), or an immunogenic fragment thereof, and which can elicit an immune response against cells that overexpress legumain.

In a particularly preferred embodiment, the DNA construct comprises a minigene encoding 2 to 5 immunogenic fragments of legumain (e.g., human legumain) linked by three-amino acid linker peptides (e.g., AAA or AAY), with one linker peptide between each immunogenic fragment.

Useful DNA constructs, including minigene constructs, preferably include regulatory elements necessary for expression of nucleotides. Such elements include, for example, a promoter, an initiation codon, a stop codon, and a polyadenylation signal. In addition, enhancers are often required for expression of a sequence that encodes an immunogenic target protein. As is known in the art, these elements are preferably operably linked to the sequence that encodes the desired protein. Regulatory elements are preferably selected that are operable in the species to which they are to be administered. Preferably, the DNA construct is in the form of a plasmid or is incorporated into a viral or bacterial vector. The DNA construct encoding legumain initially can be incorporated into a bacterial vector by transfection, using methods well known in the art. Subsequently, the transformed bacteria can be cultured to provide a ready stock of bacteria, which include legumain DNA within the genetic material of the bacteria. Cultures of such transformed bacteria provide a ready source for the DNA compositions of the present invention.

Initiation codons and stop codons are preferably included as part of a nucleotide sequence that encodes the endopeptidase or immunogenic polypeptide in a DNA composition of the present invention. The initiation and termination codons must be in frame with the coding sequence.

Promoters and polyadenylation signals included in a composition of the present invention preferably are selected to be functional within the cells of the subject to be immunized.

Examples of promoters useful in the compositions of the present invention, especially in the production of a genetic vaccine DNA composition for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes, such as human actin, human myosin, human hemoglobin, human muscle creatine, and human metalothionein.

Examples of polyadenylation signals useful in the DNA compositions of the present invention, especially in the production of a DNA composition for humans, include but are not limited to SV40 polyadenylation signals and LTR polyadenylation signals.

In addition to the regulatory elements required for DNA expression, other elements can also be included in the DNA molecule. Such additional elements include enhancers. The enhancer can be, for example, human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the species to be immunized. One having ordinary skill in the art can produce DNA constructs that are functional in a given subject species.

DNA constructs useful in the present compositions can be “naked” DNA, as defined in Restifo et al. Gene Therapy 2000; 7:89-92, the relevant disclosure of which is incorporated by reference. Preferably, the DNA construct is in the form of a plasmid or DNA that is incorporated into the genetic material of an attenuated virus or attenuated bacterium. Useful delivery vehicles or carriers include biodegradable microcapsules, immuno-stimulating complexes (ISCOMs), and liposomes for naked DNA constructs, and various physiologically acceptable buffers for genetically engineered attenuated live viruses or bacteria.

Examples of suitable attenuated live bacterial vectors that can be transformed to incorporate a FAP DNA construct include Salmonella typhimurium, Salmonella typhi, Shigella species, Bacillus species, Lactobacillus species, Bacille Calmette-Guerin (BCG), Escherichia coli, Vibrio cholerae, Campylobacter species, Listeria species, or any other suitable bacterial vector, as is known in the art. Preferably the vector is an attenuated live Salmonella typhimurium vector particularly when the composition is intended for oral administration. Preferred attenuated live Salmonella typhimurium include AroA⁻ strains such as SL7207, or doubly attenuated AroA⁻, dam⁻ strains, such as RE88.

Methods of transforming live bacterial vectors with an exogenous DNA construct are well described in the art. See, for example, Joseph Sambrook and David W. Russell, Molecular Cloning, A Laboratory Manual, 3rd Ed., Cold Spring Harbor Laboratory Press, Cold Spring Harbor, N.Y. (2001) (Sambrook and Russell). After transformation, the exogenous genetic material is incorporated into the genetic material of the bacterium, so that as the bacteria reproduce, the exogenous DNA is replicated along with the native DNA of the organism. Thus, once the bacterium has been transformed, the normal reproductive processes of the organism provides a ready supply of the exogenous DNA.

Preferred viral vectors include Bacteriophages, Herpes virus, Adenovirus, Adeno-associated virus, Sindbis virus, Polio virus, Vaccinia virus, and Avipox. Methods of transforming viral vector with an exogenous DNA construct are also well described in the art. See Sambrook and Russell, above.

Useful liposome carrier vehicles are unilamellar or multilamellar vesicles, having a membrane portion formed of lipophilic material and an interior aqueous portion. The aqueous portion is used in the present invention to contain the polynucleotide material to be delivered to the target cell. It is generally preferred that the liposome forming materials have a cationic group, such as a quaternary ammonium group, and one or more lipophilic groups, such as saturated or unsaturated alkyl groups having about 6 to about 30 carbon atoms. One group of suitable materials is described in European Patent Publication No. 0187702, and further discussed in U.S. Pat. No. 6,228,844 to Wolff et al., the relevant disclosures of which are incorporated by reference. Many other suitable liposome-forming cationic lipid compounds are described in the literature. See, e.g., L. Stamatatos, et al., Biochemistry 1988; 27:3917-3925; and H. Eibl, et al., Biophysical Chemistry 1979; 10:261-271. Alternatively, a microsphere such as a polylactide-coglycolide biodegradable microsphere can be utilized. A nucleic acid construct is encapsulated or otherwise complexed with the liposome or microsphere for delivery of the nucleic acid to a tissue, as is known in the art.

Other useful carrier vehicles include polymeric microspheres comprising biodegradable poly(ortho ester) materials, as described by Wang et al., Nat. Mater., 2004; 3(3):190-6. Epub 2004 Feb. 15, the relevant disclosures of which are incorporated herein by reference.

Preferably, the compositions embodying the present invention comprise DNA constructs that immunogenic fragments of human legumain or a functional homolog thereof. Functional homologs of legumain preferably share at least about 80% amino acid residue sequence identity with human legumain, more preferably, at least about 90%, most preferably at least about 95% identity with human legumain.

GenBank is a genetic sequence database of the National Institutes of Health (NIH), which is an annotated collection of all publicly available DNA sequences. GenBank is part of the International Nucleotide Sequence Database Collaboration, a combined effort of the DNA DataBank of Japan (DDBJ), the European Molecular Biology Laboratory (EMBL), and GenBank at the National Center for Biotechnology Information.

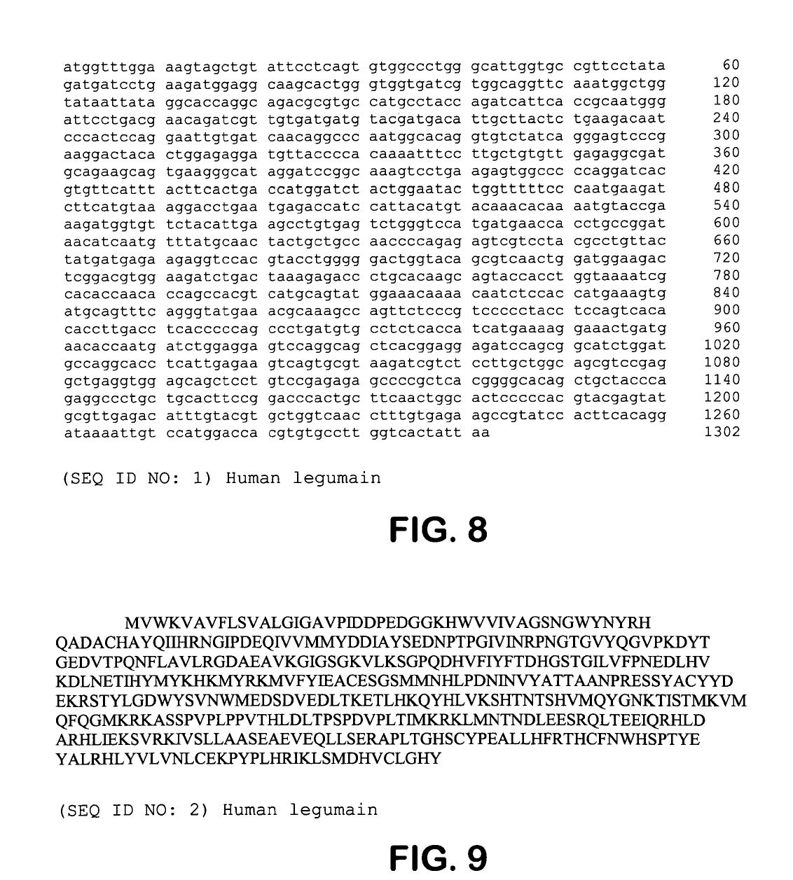

The nucleic acid sequence of a nucleic acid encoding human legumain, SEQ ID NO: 1 (FIG. 8) has been published in GenBank Accession No. BC026250, the disclosure of which is incorporated herein by reference. The corresponding amino acid residue sequence of human legumain is SEQ ID NO: 2 (FIG. 9).

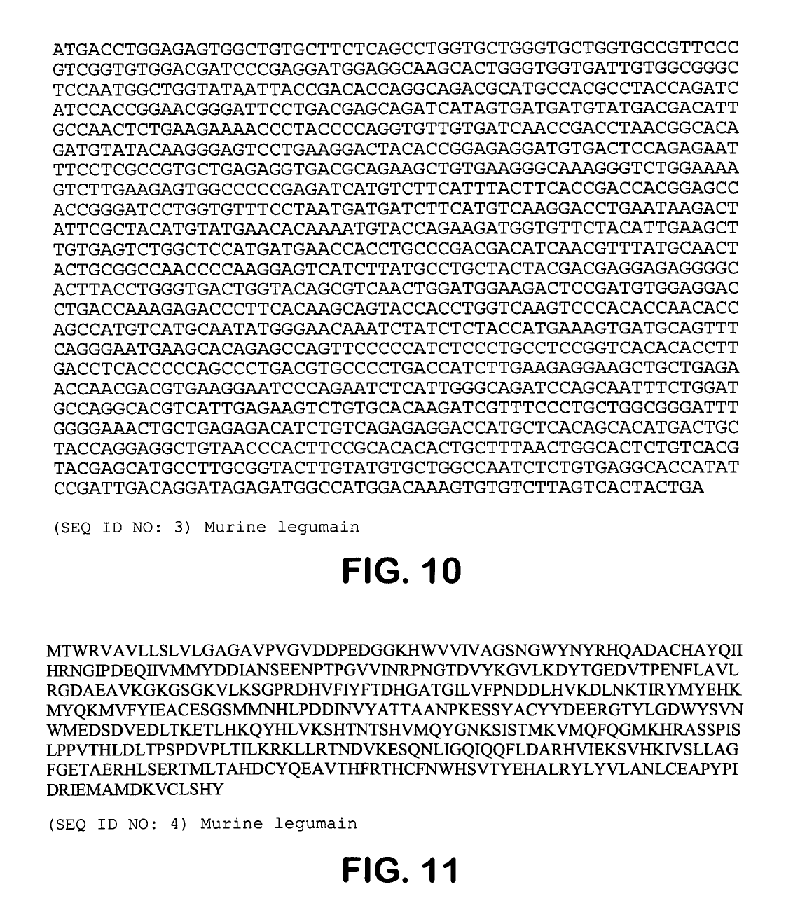

The nucleic acid sequence of a DNA encoding murine legumain, SEQ ID NO: 3 is shown in FIG. 10. The corresponding amino acid residue sequence of murine legumain is SEQ ID NO: 4 (FIG. 11).

Chen et al., J. Biological Chem. 1997, 272(12): 8090-8098 (which is incorporated herein by reference), have reported the structure and characterization of porcine legumain, which has about 83 percent sequence identity to human legumain.

Due to the inherent degeneracy of the genetic code, other DNA sequences that encode the amino acid sequence to human legumain, can be used in the practice of the invention. Such DNA sequences include those which are capable of hybridizing to human legumain as well.

DNA sequences that encode human legumain, and which can be used in accordance with the invention, include nucleic acids having deletions, additions or substitutions of different nucleotide residues for those in SEQ ID NO: 1, which result in a sequence that encodes the same legumain gene product. DNA molecules encoding for functionally equivalent homologs of human legumain can also be used in the DNA compositions of the present invention.

The gene product encoded by nucleic acid may also contain deletions, additions or substitutions of amino acid residues within the legumain amino acid residue sequence, which results in a silent change, thus producing a functionally equivalent legumain. Such amino acid substitutions may be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. As used herein, a functionally equivalent legumain refers to protein that includes one or more epitopes, which when recognized by T cells, allow these same T cells to recognize legumain epitopes displayed on legumain-expressing cells. In preferred embodiments, a functionally equivalent legumain has an amino acid residue sequence that has at least about 80% sequence identity to the amino acid residue sequence of human legumain (SEQ ID NO: 2), e.g., at least about 90% sequence identity, or at least about 95% sequence identity.

The DNA constructs encoding legumain can be engineered in order to alter the legumain coding sequence (relative to the native legumain DNA, SEQ ID NO: 1) for a variety of ends including, but not limited to, alterations that modify processing and expression of the legumain gene product. For example, mutations may be introduced in the DNA using techniques that are well known in the art, e.g., site-directed mutagenesis, to insert new restriction sites, to alter glycosylation patterns, phosphorylation, etc.

In one preferred embodiment, the DNA composition of the invention comprises a DNA construct encoding an immunogenic polypeptide comprising a plurality of immunogenic fragments of human legumain and a DNA construct operably encoding at least one immune effector protein, both of which are expressible in immune cells. As used herein and in the appended claims the phrase “immune effector protein” means a protein that is involved in regulation of an immune system pathway. Preferably, the immune effector protein is a cytokine.

Cytokines are proteins and polypeptides produced by cells that can affect the behavior of other cells, such as cell proliferation, cell differentiation, regulation of immune responses, hematopoiesis, and inflammatory responses. Cytokines have been classified into a number of families, including chemokines, hematopoietins, immunoglobulins, tumor necrosis factors, and a variety of unassigned molecules. See generally Oxford Dictionary of Biochemistry and Molecular Biology, Revised Edition, Oxford University Press, 2000; and C. A. Janeway, P. Travers, M. Walport and M. Schlomchik, Immunobiology, Fifth Edition, Garland Publishing, 2001 (hereinafter “Janeway and Travers”). A concise classification of cytokines is presented in Janeway and Travers, Appendix III, pages 677-679, the relevant disclosures of which are incorporated herein by reference.

Hematopoietins include, for example erythropoietin, interleukin-2 (IL-2, a 133 amino acid protein produced by T cells and involved in T cell proliferation), IL-3, IL-4, IL-5, IL-6, IL-7, L-9, IL-11, IL-13, IL-15 (a 114 amino acid IL-2-like protein, which stimulates the growth of intestinal epithelium, T cells, and NK cells), granulocyte colony-stimulating factor (G-CSF), granulocyte-macrophage colony-stimulating factor (GM-CSF), oncostatin M (OSM), and leukemia inhibitory factor (LIF).

Interferons include, for example, IFN-α, IFN-β, and IFN-γ (a 143 amino acid homodimeric protein produced by T cells and NK cells, which is involved in macrophage activation, increased expression of MHC molecules and antigen processing components, IG class switching, and suppression of T_{H}2).

Immunoglobulins include, for example, B7.1 (CD80), and B7.2 (CD86), both of which co-stimulate T cell responses.

The tumor necrosis factor (TNF) family includes, for example, TNF-α, TNF-β (lymphotoxin), lymphotoxin-β(LT-β), CD40 ligands, Fas ligand, CD27 ligand, CD30 ligand, 4-1BB ligand, Trail, and OPG ligand.

The biological roles of CD40 ligand (CD40L), particularly its interaction with CD40 expressed on antigen presenting cells during costimulation of T cell activation, are well known in the art. CD40 is a 48 kDa glycoprotein expressed on the surface of all mature B cells, most mature B-cell malignancies, and some early B-cell acute lymphocytic leukemias, but it is not expressed on plasma cells, Clark, Tissue Antigens 1990, 35:33-36. CD40L, a type II membrane protein of about 35 kDa, is expressed on the surface of T cells upon antigen recognition. Members of the TNF family are biologically most active when expressed as homotrimers. CD40L is no exception in this regard and can be expressed as a homotrimer (CD40LT) by modification of a 33 amino acid leucine zipper motif fused to the N-terminus of the entire extracellular domain of this ligand. CD40LT DNA has been reported by Gurunathan et al. J. Immunol. 1998, 161:4563, to enhance cellular immune responses such as induction of IFN-γ and cytolytic T cell activity when mice were vaccinated with DNA encoding the highly immunogenic model antigen β-galactosidase.

CD40LT is an important factor in the activation of T cells necessary to induce an effective protective immunity against tumor self-antigens. Once MHC class I antigen:peptide complexes are taken up by dendritic cells (DCs) and presented to naive T cells, the first antigen signal is delivered via T cell receptors (TCR), followed by upregulation of CD40LT. On the T cell surface, CD40LT then induces costimulatory activity on DCs via CD40-CD40LT interactions. Thus primed, these antigen presenting cells can express costimulatory molecules B7.1 (CD80) and B7.2 (CD86), which sends a second costimulatory signal to T cells via interaction with CD28, an event required for full activation of T cells to concurrently produce pro-inflammatory cytokines INF-γ and IL12, and to perform effector functions.

Various cytokines that are not assigned to a particular family include, for example, tumor growth factor-β (TGF-β), IL-1α, IL-1β, IL-1 RA, IL-10, IL-12 (natural killer cell stimulatory factor; a heterodimer having a 197 amino acid chain and a 306 amino acid chain, which is involved in NK cell activation and induction of T cell differentiation to T_{H}1-like cells), macrophage inhibitory factor (MIF), IL-16, IL-17 (a cytokine production-inducing factor, which induces cytokine production in epithelia, endothelia, and fibroblasts), and IL-18.

Chemokines are a family of cytokines that are relatively small chemoattractant proteins and polypeptides, which stimulate the migration and activation of various cells, such as leucocyte migration (e.g., phagocytes and lymphocytes). Chemokines play a role in inflammation and other immune responses. Chemokines have been classified into a number of families, including the C chemokines, CC chemokines, CXC chemokines, and CX₃C chemokines. The names refer to the number and spacing of cysteine (c) residues in the molecules; C chemokines having one cysteine, CC chemokines having two contiguous cysteines, CXC having two cysteines separated by a single amino acid residue, and CX₃C chemokines having two cysteines separated by three amino acid residues. Chemokines interact with a number of chemokine receptors present on cell surfaces. See Janeway and Travers, Appendix IV, page 680, which is incorporated herein by reference.

In addition, chemokines can have immunomodulating activity and have been implicated in immune responses to cancer. For example, murine 6Ckine/SLC, the mouse analog of the human secondary lymphoid tissue chemokine (SLC), now commonly referred to as CCL21, has been reported to induce an antitumor response in a C-26 colon carcinoma tumor cell line. See Vicari, et al. J. Immunol. 2000; 165(4):1992-2000. Human CCL21 and its murine counterpart, 6Ckine/SLC, are classified as CC chemokines, which interact with the CCR7 chemokine receptor. Murine 6Ckine/SLC (muCCL21) is also reported by Vicari et al. to be a ligand for the CXCR3 chemokine receptor. Human CCL21, murine muCCL21 and a variety of other chemokines are implicated in the regulation of various immune system cells such as dendritic cells, T cells, and natural killer (NK) cells.

Mig and EP-10 are CXC chemokines that interact with the CXCR3 receptor, which is associated with activated T cells. Lymphotactin is a C chemokine, which interacts with the XCR1 receptor associated with T cells and NK cells. Fractalkine is a CX₃C chemokine, which interact with the CX₃CR1 receptor that is associated with T cells, monocytes and neutrophils.

Particularly preferred immune effector proteins to be encoded by the DNA compositions of the present invention include cytokines IL-2 (a hematopoietin), CCL21 (a chemokine), as well as CD40 ligands such as CD40 ligand trimer (CD40LT), a TNF family cytokine.

DNA and protein sequences for human IL-2 have been published in GenBank, Accession No. BC070338, the disclosures of which are incorporated herein by reference. The DNA and protein sequences of murine IL-2 have been in GenBank, Accession No. NM 008366, the disclosures of which are incorporated herein by reference.

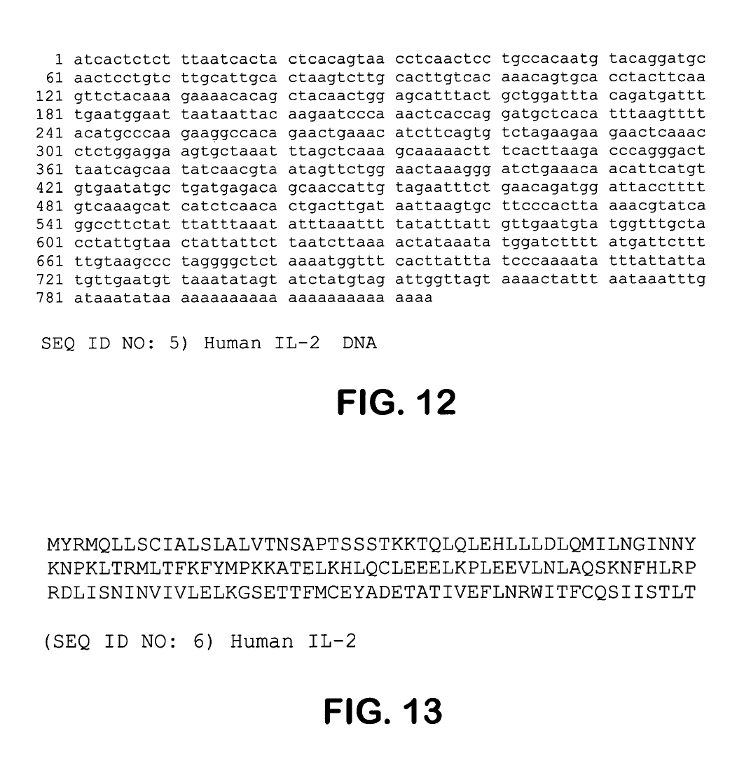

The nucleic acid sequence encoding human IL-2 is presented in FIG. 12 (SEQ ID NO: 5), and its corresponding amino acid residue sequence (SEQ ID NO: 6) is provided in FIG. 13.

DNA and protein sequences for human CCL21 have been published in GenBank, Accession No. AB002409, the disclosures of which are incorporated herein by reference.

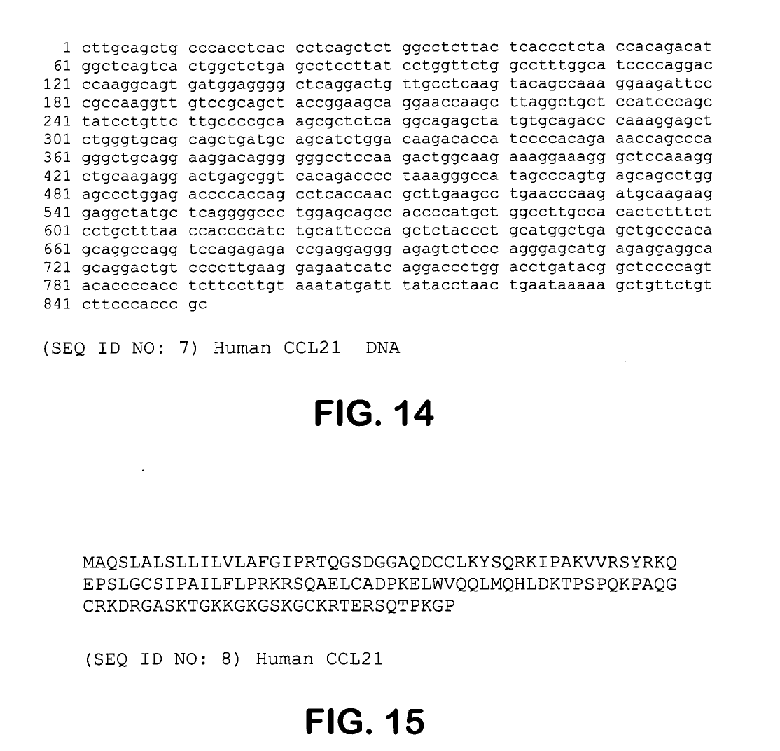

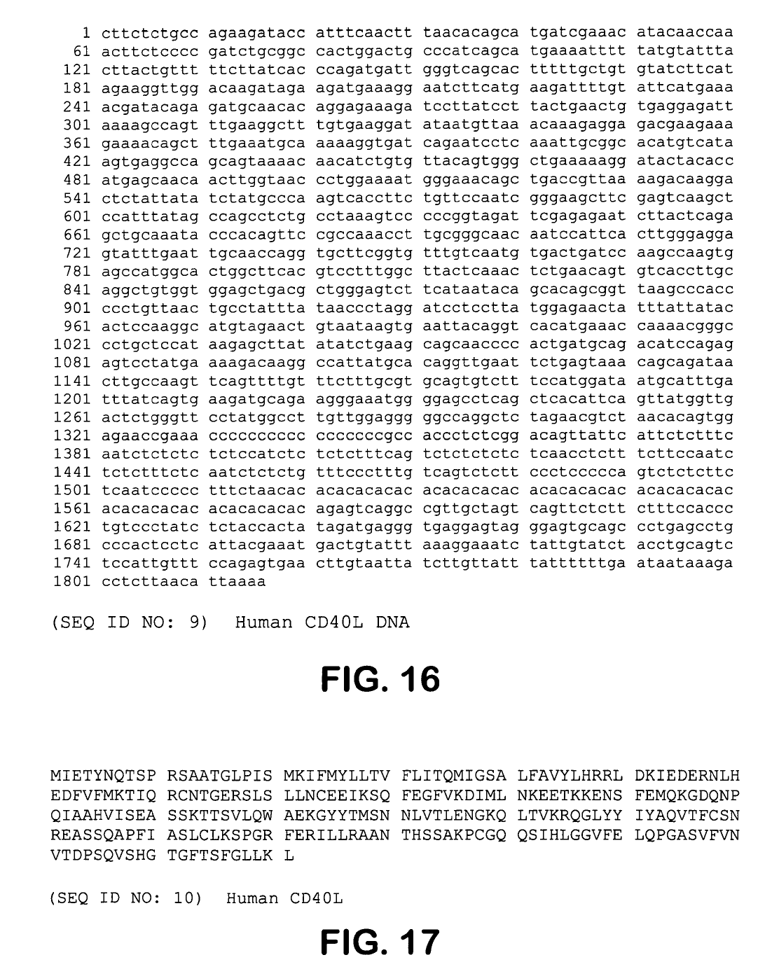

The nucleic acid sequence encoding human CCL21 is presented in FIG. 14 (SEQ ID NO: 7), and its corresponding amino acid residue sequence (SEQ ID NO: 8) is provided in FIG. 15.

Human CD40 ligand (CD40L) is a 261 amino acid protein, which exists as a trimer (CD40LT) in its most active form. The DNA sequence encoding human CD40L (also known as CD 154) has been published in GenBank, Accession No. NM 000074, the disclosure of which is incorporated herein by reference (FIG. 16, SEQ ID NO: 9). The corresponding protein sequence of CD40L is shown in FIG. 17 (SEQ ID NO: 10).

The method aspects of the present invention involve administering to a mammal a DNA composition comprising a DNA construct encoding legumain, which is expressible in immune cells of the mammal. Preferably, the mammal is a human. The composition can be administered orally, intramuscularly, intranasally, intraperitoneally, subcutaneously, intradermally, or topically, depending upon the particular dosage form in which the composition is prepared. Preferably the composition is prepared in an orally administrable dosage form, such as a solution, suspension, emulsion, capsule, tablet, and the like.

A DNA composition of the invention can be utilized to provide long term inhibition of tumor growth and/or tumor metastases in a patient treated with the composition. In a preferred embodiment, the DNA composition is administered in conjunction with an antitumor chemotherapeutic agent. The DNA composition can be administered together with the chemotherapeutic agent in a combined dosage form, or the composition and chemotherapeutic agent can be administered in separate dosage forms and at separate dosage intervals tailored to the pharmacology of the chemotherapeutic agent being administered.

Chemotherapeutic agents useful in combination with the DNA compositions of the present invention include antitumor agents such as doxorubicin, paclitaxol, a cyclophosphamide, etoposide, 5-fluorouracil, methotrexate, and the like.

DNA compositions of the present invention are preferably formulated with pharmaceutically acceptable carriers or excipients such as water, saline, dextrose, glycerol, and the like, and combinations thereof to aid in formulation and administration of the composition. The compositions can also contain auxiliary substances such as wetting agents, emulsifying agents, buffers, and other auxiliary substances that are well known in the pharmaceutical arts.

The compositions of the present invention are preferably administered orally to a mammal, such as a human, as a solution or suspension in a pharmaceutically acceptable carrier, at a DNA concentration in the range of about 1 to about 10 micrograms per milliliter, based on the weight of the DNA that encodes legumain. A particularly preferred dosage form for a DNA composition of the invention is a suspension of attenuated legumain-transfected bacteria in a suitable buffer solution, which can be formulated for oral administration. The appropriate dosage of the composition will depend upon the subject to be vaccinated, on the activity of the composition, and in part upon the judgment of the medical practitioner administering or requesting administration of the composition.

The dosage to be administered to the mammal, and the schedule of administration if more than one administration is to be used, will vary from mammal to mammal and upon the dosage form. Effective dosage amounts and administration schedules can be determined empirically through clinical dose-response studies, as is well known in the art. The dosage and dosage schedule is selected to provide a sufficient amount of legumain expression in immune cells to elicit an immune response in the mammal against tumor-associated macrophages that express legumain. Preferably, the dosage of the composition administered to the subject mammal elicits expression of a sufficient amount of legumain antigen in immune cells of the mammal to sustain an immune response against legumain-presenting tumor-associated macrophages that will continue over a period of at least a month, e.g., at least 6 months or at least about one year. In some preferred embodiments, the dosage of legumain transformed cells administered to the patient is about 1×10⁸ cells transfected with about 0.3 to about 0.8 μg of legumain DNA.

The compositions of the present invention can be packaged in suitably sterilized containers such as ampules, bottles, or vials, either in multi-dose or in unit dosage forms. The containers are preferably hermetically sealed after being filled with a DNA composition of the invention. Preferably, the compositions are packaged in a container having a label affixed thereto, which label identifies the composition, and bears a notice in a form prescribed by a government agency such as the United States Food and Drug Administration reflecting approval of the composition under appropriate laws, dosage information, and the like. The label preferably contains information about the composition that is useful to an health care professional administering the composition to a patient. The package also preferably contains printed informational materials relating to the administration of the composition, instructions, indications, and any necessary required warnings.

The following examples are provided to further illustrate the features and embodiments of the present invention, and are not meant to be limiting.

### Materials and Methods.

Animals, Bacterial Strains and Cell Lines. Female BALB/c and C57BL/6 mice, 6-8 weeks of age, were purchased from The Scripps Research Institute Rodent Breeding Facility. The double attenuated S. typhimurium strain RE88 (aroA⁻, dam⁻) was obtained from Remedyne Corporation, Goleta, Calif. The murine CT-26 colon cancer cell line was kindly provided by Dr. I. J. Fidler (MD Anderson Cancer Center) and the murine D121 non-small cell lung carcinoma cells were a gift from Dr. L. Eisenbach (Weizmann Institute of Science, Rehovot, Israel). The murine 4T1 breast carcinoma cells were kindly provided by Dr. Suzanne Ostrand-Rosenberg (University of Maryland).

Immunohistochemical analyses. Immunohistochemical analyses were performed on 4T1 tumor tissues and Matrigel plug sections. Legumain expression of macrophages was identified on 4T1 tumor tissue sections with biotinylated rat anti-mouse CD68 mAb (BD Bioscience Pharmingen) with GFP-conjugated streptavidin being the secondary reporter reagent. Rabbit anti-legumain antiserum was prepared by immunization with purified human legumain produced in E. coli. (Ishii, Methods Enzymol. 1994; 244:604-615). The reaction was visualized with Texas-red conjugated streptavidin. Additionally, 4T1 tumor tissue sections and Matrigel plug sections were fixed and stained with MMP-9, VEGF, TGF-beta and F4/80 antibodies (eBioscience, San Diego, Calif.) in 4T1 tumor tissue section while CD68 and CD31 antibodies (BD Bioscience Pharmingen) were used in Matrigel plug sections. All tissue sections were visualized with Texas red or GFP conjugated streptavidin as the secondary reporting reagent, and the slides analyzed with laser scanning by confocal microscopy (Bio-Rad Laboratories). All the images were captured by a SPOT™ cooled color digital camera system (Diagnostic Instruments. Inc).

Immunization and Tumor Cell Challenge. Prophylactic model: BALB/c or C57BL/6 mice were each divided into three experimental groups (n=8) and immunized with PBS, empty vector or pUb-legumain transfected S. typhimurium. All mice were challenged by intravenous (i.v.) injection with about 5×10⁴ CT-26 cells (BALB/c), about 2×10⁵ D121 cells (C57BL/6) or mammary gland fat pad injection with about 7×10³ 4T1 cells (BALB/c), 1 week after the last immunization, to induce either experimental or spontaneous pulmonary metastases. The lung weights in experimental and control groups were determined 24 days after tumor cell challenge. Therapeutic model: BALB/c mice were divided into three experimental groups (n=8) and first injected into the fat-pad with about 7×10³ 4T1 cells on day 0 and then immunized 3 times with the DNA composition of the invention starting on days 3, 7 and 11. After 24 days, the primary tumor was excised to determine mouse lung weights and metastasis scores, or mouse survival rates.

In vivo depletion of CD4⁺ or CD8⁺ T cells, Cytotoxicity and ELISPOT assays. The depletion of CD4⁺ or CD8⁺ T cells in vivo was performed as previously described (Ceredig et al. 1985, Nature 314:98-100). Cytotoxicity was measured and calculated by a standard ⁵¹Cr-release assay as previously reported (Zhou et al. 2005, Blood 106:2026-2032). ELISPOT assays were performed with an ELISPOT kit (BD Bioscience Pharmingen) according to instructions provided by the manufacturer.

In vivo Matrigel angiogenesis assay. Matrigel was used for evaluating the suppression of angiogenesis after vaccination. Briefly, BALB/c mice were injected subcutaneously (s.c.) 2 weeks after the last vaccination, into the sternal region with growth factor-reduced Matrigel (BD Bioscience) containing VEGF or bFGF-2 (about 200 ng/plug) and 4T1 tumor cells (about 5×10³/plug) that were previously irradiated with 1000 Gray (about 100,000 rad) of gamma radiation. The endothelium was stained 6 days after Matrigel implantation by i.v. injection with Bandiera simplofica lectin I (Isolectin B4), conjugated with fluorescein (Vector Laboratories). This was done along with staining the endothelium of control animals. About 30 minutes later, mice were sacrificed, Matrigel plugs extracted, and fluorescence evaluated by fluorimetry. Additionally, the Matrigel plugs were removed 6 days after Matrigel implantation, fixed in Bouin's solution for 24 hours, and then embedded in paraffin. All tissues were sectioned, mounted onto slides, and stained with Masson's trichrome. All of the images were captured by a SPOT™ cooled color digital camera system as described above.

Flow cytometry (FACS). Activation markers of T cells were measured by two-color flow cytometric analysis with a BD Biosciences FACSCALIBUR® analyzer. DC cell markers were determined by staining freshly isolated lymphocytes from successfully vaccinated mice and control mice with anti-CD11c Ab in combination with FITC-conjugated anti-CD40, CD80 and Abs against MHC-Class II Ag. Macrophages bearing high levels of CD206 and F4/80 were quantified by two-color flow analysis. Tumor cells were isolated from successfully vaccinated BALB/c mice and then stained with anti-CD206 Ab conjugated with PE (Cell Science, Inc.), anti-F4/80 Ab conjugated with APC and anti-legumain Ab conjugated with FITC, followed by FACS analyses. All antibodies were purchased from Pharmingen, San Diego, Calif. IFN-γ release at the intracellular level was determined in lymphocytes of Peyer's patches obtained 3 days after one time immunization, and stained with APC-tagged anti-CD8 Ab. Cells were fixed, permeabilized, and subsequently stained with PE-labeled anti-IFN-γ Ab to detect intracellular expression of IFN-γ.

Migration Assay. Cell migration assays were performed by using modified Boyden chambers (Transwell, Corning Inc., Corning, N.Y.). The tumor cells were harvested from tumor tissue of either treated or control groups of mice to perform a trans-well migration assay. After culturing for 4 hours the cells on the lower surface of wells were fixed with 1% paraformaldehyde, stained with 1% crystal violet and counted (Shi et al. 2004, Mol. Cancer. Res. 2:395-402).

Statistical Analysis. The statistical significance of differential findings between experimental groups and controls was determined by Student's t test. Findings were regarded as significant if two tailed P values were <0.05. Kaplan-Meier analysis was used to evaluate the survival of mice.

### Example 1

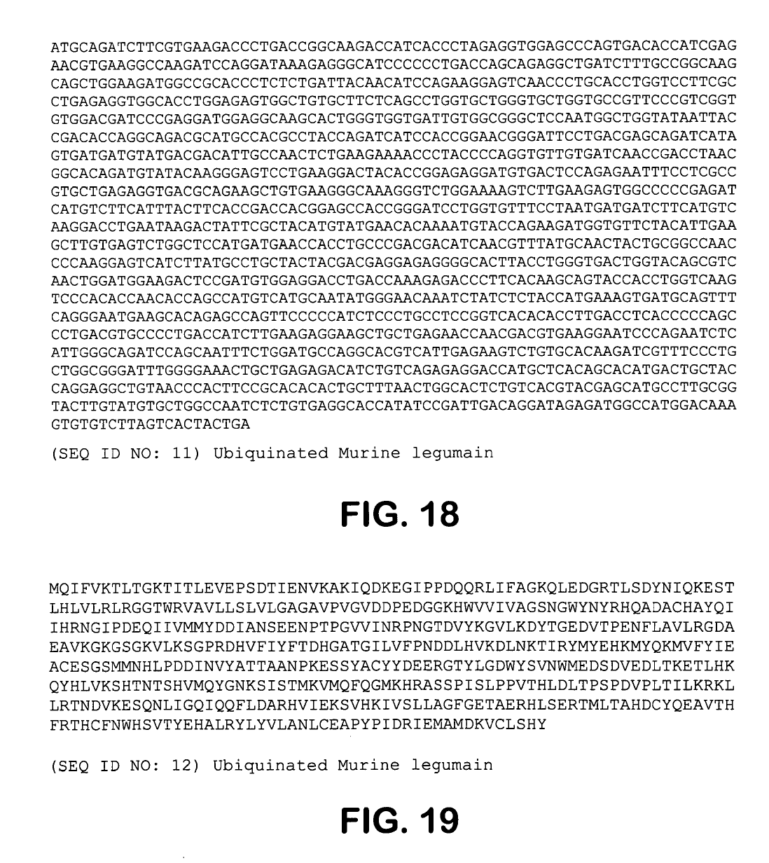

Vector construction, protein expression and transformation of S. typhimurium with DNA Plasmids. Two constructs were prepared based on a pCMV vector, which is commercially available from Invitrogen, Carlsbad, Calif. The pUb-legumain construct included polyubiquitinated, full-length murine legumain. Full-length legumain murine legumain DNA has the nucleotide sequence shown in FIG. 10, SEQ ID NO: 3 (the amino acid residue sequence of murine legumain is shown in FIG. 11, SEQ ID NO:4). An empty vector construct served as a control. The murine legumain was collected from 4T1 breast cancer cells using total RNA as a template by PCR. An expression vector was established based on the pCMV-cyto vector (Invitrogen) containing the polyubiquitin sequence cloned in front of the legumain sequence. The nucleic acid sequence of the polyubiquinated murine legumain is shown in FIG. 18 (SEQ ID NO: 11). The amino acid residue sequence of ubiquinated murine legumain is shown in FIG. 19 (SEQ ID NO: 12). Protein expression of legumain was demonstrated by Western blotting with a polyclonal rabbit anti-murine legumain Ab as well as anti-murine beta-actin Ab (Santa Cruz Biotechnology, Inc) as a loading control. The specific protein was detected with a goat-anti-rabbit-HRP conjugated IgG Ab (Bio-Red Laboratories). Attenuated Salmonella typhimurium were transduced with DNA vaccine plasmids by electroporation as described in Luo et al. 2003, Proc. Natl. Acad. Sci. U.S.A 100:8850-8855 and Xiang et al. 2000, Proc. Natl. Acad. Sci. U.S.A 97:5492-5497.

### Example 2

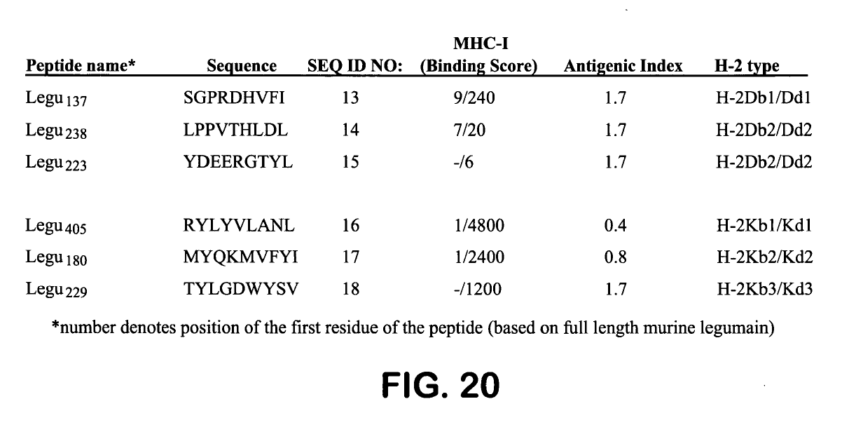

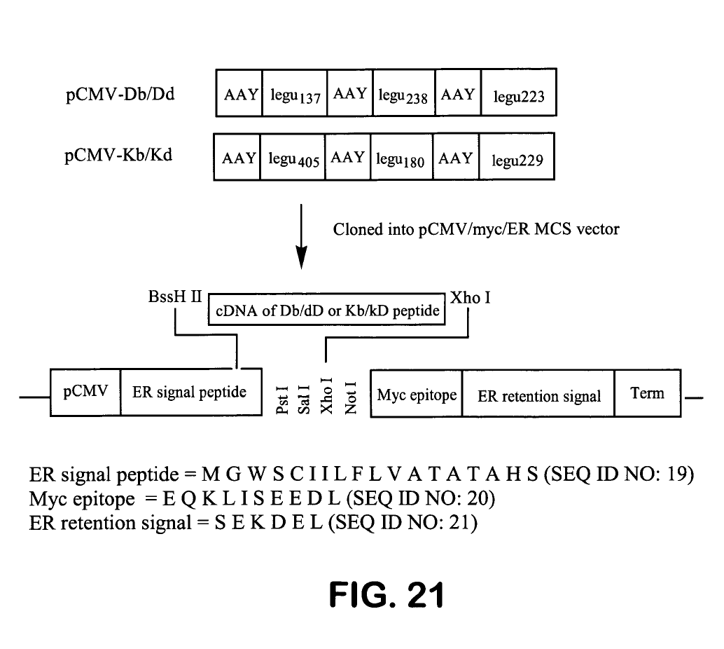

Immunogenic murine legumain fragments. Two plasmids, each comprising a legumain minigene encoding three immunogenic legumain fragments joined together by a three-amino acid spacer (AAY) between each fragment, were prepared by inserting the legumain minigene into a pCMV/myc/ER MCS plasmid, which is commercially available from Invitrogen, Carlsbad, Calif. (See FIGS. 20 and 21). The vector includes a segment encoding an ER signal peptide, a myc epitope and an ER retention signal (see FIG. 21). The insertion of the minigene was made between a BssH II site in the ER signal peptide segment and a Xho I site, as shown in FIG. 21. The first legumain minigene plasmid (pCMV-Db/Dd; also referred to a pH-2Dd in the Figures) comprises an AAY spacer, immunogenic legumain fragment legu₁₃₇, an AAY spacer, immunogenic legumain fragment legu₂₃₈, an AAY spacer, and immunogenic legumain fragment legu₂₂₃. The second legumain minigene plasmid (pCMV-Kb/Kd; also referred to a pH-2 Kd in the Figures) comprises an AAY spacer, immunogenic legumain fragment legu₄₀₅, an AAY spacer, immunogenic legumain fragment legu₁₈₀, an AAY spacer, and immunogenic legumain fragment legu₂₂₉. FIG. 20 shows the amino acid residue sequences for the immunogenic legumain fragments, as well as the MHC class I binding scores and sequence identifier number (SEQ ID NO) for each fragment. Peptide expression was verified by Western blotting of transfected COS-7 cells with monoclonal anti-myc antibody (Invitrogen). Once peptide expression was verified, a stop codon was introduced immediately in front of the myc epitope sequences of the vector. The resulting minigene plasmid vectors (pCMV-Db/Dd and pCMV-Kb/Kd) were each verified by nucleotide sequencing and then were transfected into attenuated S. typhimurium RE88 bacteria by electroporation to afford DNA compositions of the invention encoding legumain minigenes. As described in detail below, these compositions were effective for attacking 4T1 and D2F2 breast carcinoma in Balb/c mice. The observed tumor protective response was mediated by CD8 T cells, which specifically killed legumain⁺ tumor-associated macrophage cells, resulting in a marked suppression of tumor angiogenesis.

Oral Immunization and Tumor Cell Challenge. Groups of BALB/c mice (n=8) were treated 3 times at 1-week intervals by oral gavage with 100 μl PBS containing approximately 5×10⁸ CFU of doubly attenuated S. typhimurium harboring either empty vector, the pCMV-Db/Dd plasmid, or the pCMV-Kb/Kd plasmid. Mice were challenged i.v. or s.c. with D2F2 carcinoma cell lines 2 weeks after the last treatment.

Cytotoxicity Assay. Cytotoxicity was measured by a standard ⁵¹Cr-release assay as previously described. The percentage of specific target cell lysis was calculated by the formula [(E−S)/(T−S)]×100, where E is the average experimental release, S the average spontaneous release, and T is the average total release.

ELISPOT Assay. Splenocytes were collected 2 weeks after D2F2 tumor cell challenge from all experimental groups of BALB/c mice, and cultured for 24 hours with either irradiated (1000 Gy) 4T1 cells or 4T1 cells obtained from freshly harvested 4T1 breast tumor tissue. The assay was performed according to instructions provided by the manufacturer (BD Bioscience, San Jose, Calif.).

Evaluation of anti-angiogenic activity. Suppression of angiogenesis was determined by the Matrigel assay as previously described. Vessel growth in the Matrigel was determined by measuring the concentration of hemoglobin using Drabkin's reagent (aqueous solution containing 1 g/L NaHCO₃, 0.05 g/L KCN, and 0.2 g/L K₃Fe(CN)₆). Matrigel plugs were removed 6 days after Matrigel implantation, fixed in Bouin's solution (15 parts w/w saturated aqueous picric acid, 5 parts w/w of 37% aqueous formalin, and 1 part w/w of glacial acetic acid) for 24 hours, and embedded in paraffin. All tissues were sectioned, mounted onto slides and stained with Masson's trichrome. All images were captured by the SPOT™ Cooled Color digital camera system. Using the minigene approach of the present invention, the syngeneic BALB/c mice, anti-TAM minigene DNA compositions suppressed both tumor growth and angiogenesis. Six legumain immunogenic peptides were evaluated as H-2D^{d}-1,2,3, or H-2 K^{d}-1,2,3-restricted minigenes, based on the binding prediction of these MHC class I antigen molecules by the HLA Peptide Binding Predictions program provided by the BioInformatics & Molecular Analysis Section (BIMAS) of NIH website. The amino acid sequences of these peptides and their binding activities, as predicted by DNASTAR® software (DNAStar, Inc., Madison, Wis.), are listed in FIG. 20.

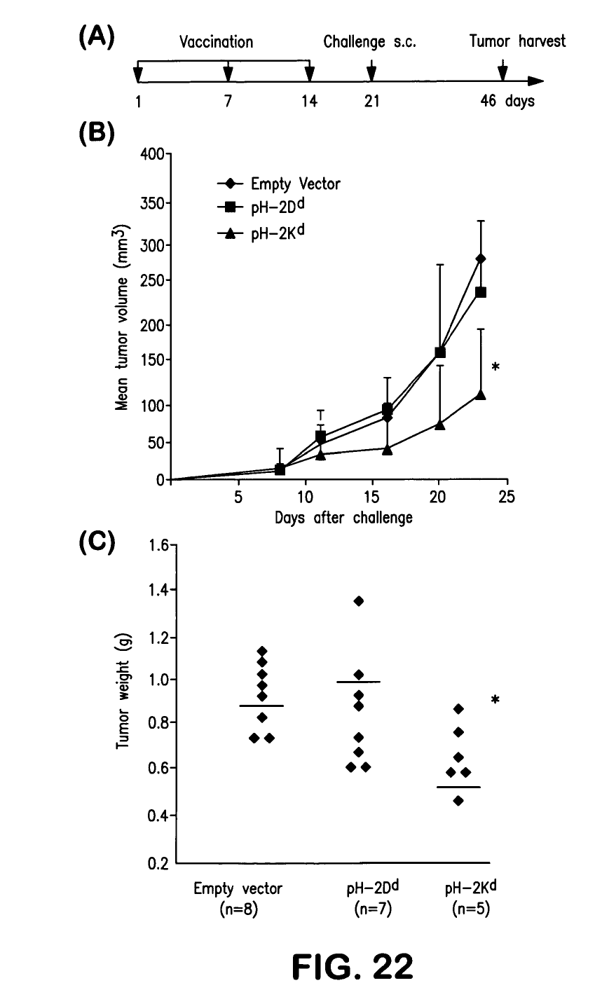

The pCMV-Kb/Kd Minigene vaccine protects mice against D2F2 breast tumor cell challenge and prevents pulmonary metastasis. Initially, the minigene DNA compositions were tested in prophylactic breast carcinoma model, where mice were first vaccinated with the minigene composition and then challenged s.c., with murine D2F2 breast carcinoma cells (FIG. 22). A marked inhibition of tumor growth was observed in syngeneic BALB/c mice vaccinated with pCMV-Kb/Kd, but not with pCMV-Db/Dd. In contrast, all mice vaccinated with only the empty vector control vector revealed rapid s.c. tumor growth.

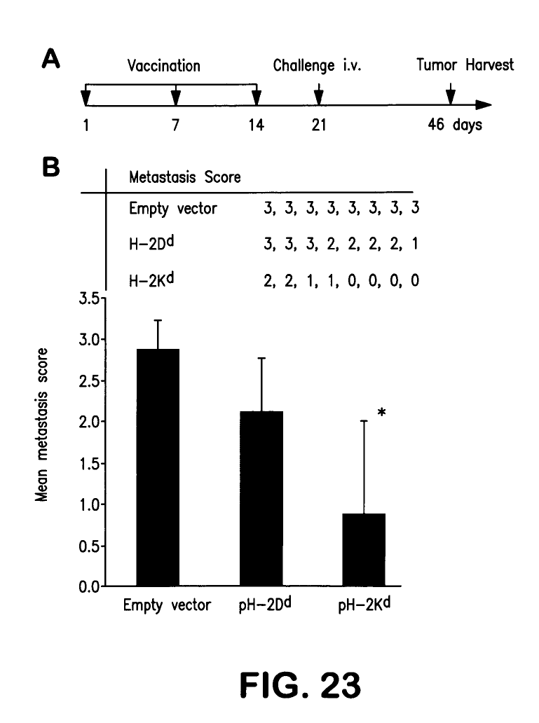

The pCMV-Kb/Kd legumain minigene vaccine suppressed pulmonary D2F2 metastasis by attacking TAMs as indicated by the marked inhibition of experimental metastases observed in BALB/c mice challenged by i.v. injection of D2F2 breast carcinoma cells 2 weeks after the third vaccination. In contrast, pCMV-Db/Dd did prevent metastases in this model. Mice vaccinated with only the empty vector control revealed uniform, rapid metastatic pulmonary tumor growth (FIG. 23).

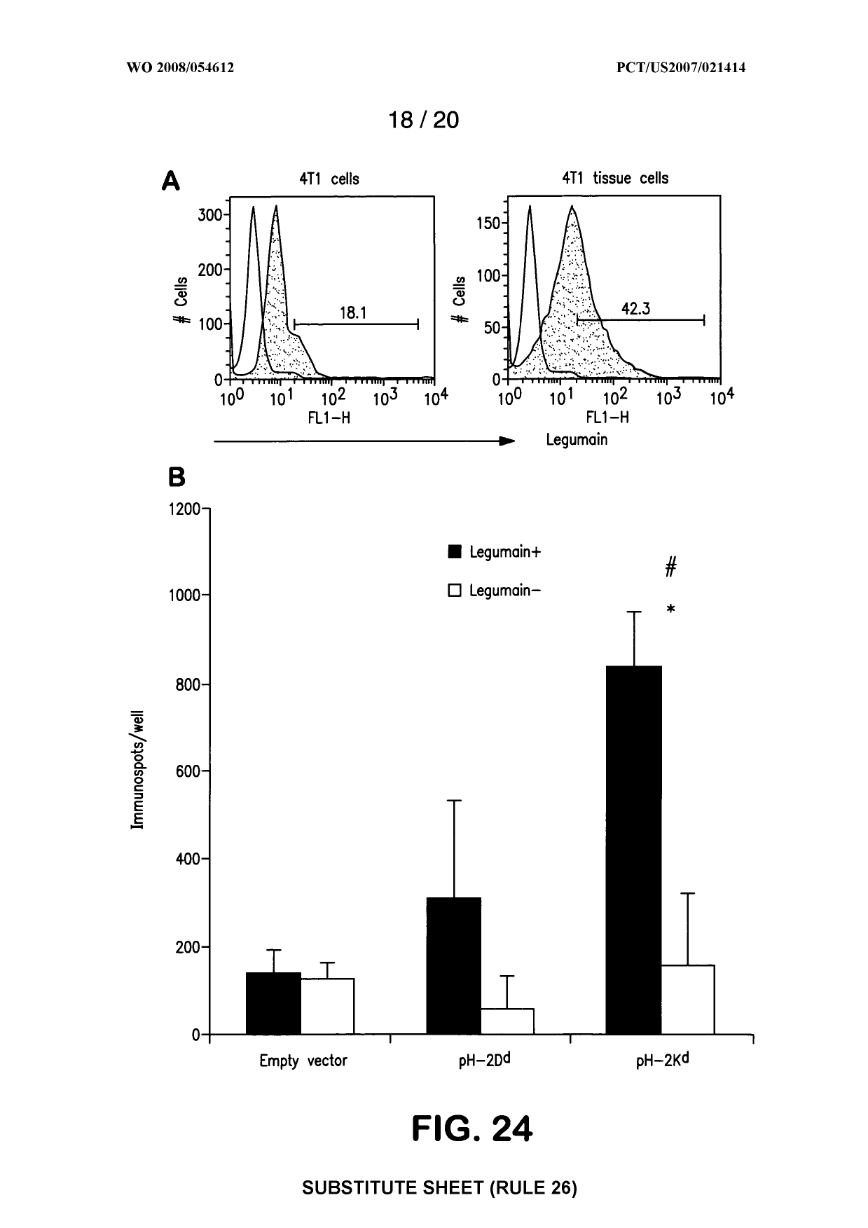

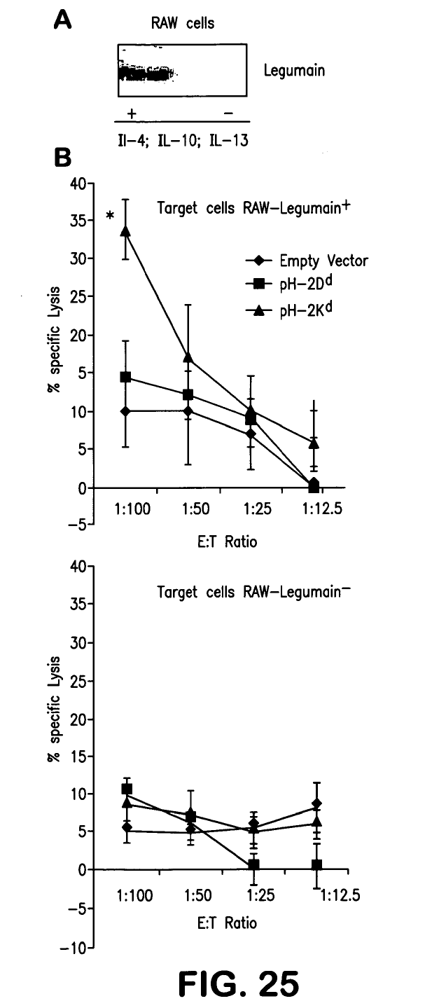

The minigene vaccine induces a CTL response which is capable of killing legumain⁺ cells. In order to delineate the specific T cells response achieved after minigene vaccinations, T cell activation was demonstrated by the specific release of IFN-γ by activated T cells (FIG. 24, Panel B). These cells were stimulated with cells harvested from fresh 4T1 tumor tissue that highly expressed legumain as indicated by flow cytometry analysis (FIG. 24, Panel A). Importantly, cytotoxicity assays clearly demonstrated marked CTL activity only in immunized mice. The target cells were cells of murine marcophage cell line RAW, which had been cultured with IL-4, IL-10 and IL-13 cytokines to induce the expression of legumain. This was also verified by Western blot analysis (FIG. 25, Panel A), where wild type RAW cells were shown to be legumain-negative. Splenocytes isolated from control mice treated with empty vector showed similar background killing of RAW cells, either positive or negative for legumain expression (FIG. 25, Panel B). However, splenocytes from pCMV-Kb/Kd-treated mice induced significantly stronger killing against legumain⁺ target cells than against such cells harvested from mice treated with pCMV-Db/Dd or the empty vector (FIG. 25, Panel B). These data demonstrate the specificity of the pCMV-Kb/Kd minigene-induced CTL activity and its capability to specifically kill legumain⁺ macrophages.

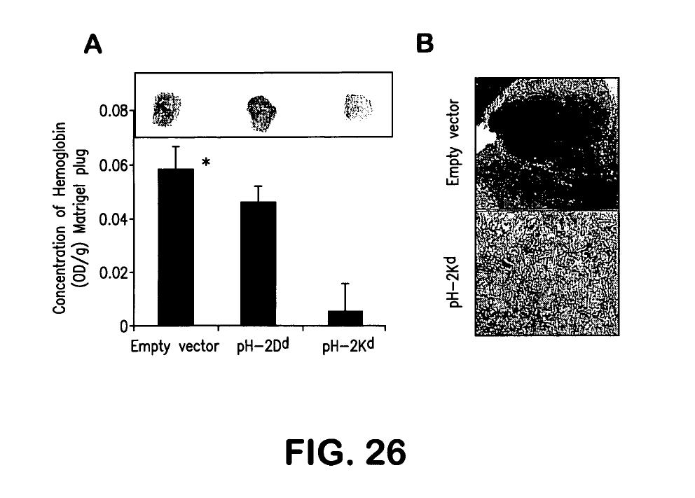

The pCMV-Kb/Kd minigene vaccine induces anti-angiogenesis effects. In order to evaluate the extent to which anti-angiogenesis plays a key role in the pCMV-Kb/Kd-vaccine-induced tumor protection, Matrigel assays were performed in which blood vessel formation was induced within the Matrigel by recombinant bFGF. The difference in vessel formation between the various treatment groups was quantified by measuring the relative concentration of hemoglobin (Hb) in Matrigel plug extracts obtained from either immunized or control mice. Thus, mice treated with the pCMV-Kb/Kd vaccine displayed a clear reduction in the average relative concentration of Hb (FIG. 26, Panel A). Furthermore, when Matrigel sections from immunized and control mice were analyzed by Masson's trichrome staining, those obtained from mice in the empty vector control group revealed ample, multiple blood vessels. In contrast, blood vessels were markedly reduced in Matrigel sections from pCMV-Kb/Kd-treated mice (FIG. 26, Panel B). These data demonstrate that immunization with the pCMV-Kb/Kd vaccine resulted in the reduction of tumor vasculature. Taken together, these findings indicate that the pCMV-Kb/Kd minigene composition induced marked anti-angiogenic effects, which aided in the protection of BALB/c mice from challenges with D2F2 breast tumor cells in a prophylactic setting.

In separate experiments, with D121 non-small cell carcinoma cells in syngeneic C57/BL mice, both the pCMV-Kb/Kd and pCMV-Db/Dd compositions provided effective anti-TAM immune responses.

### Discussion.

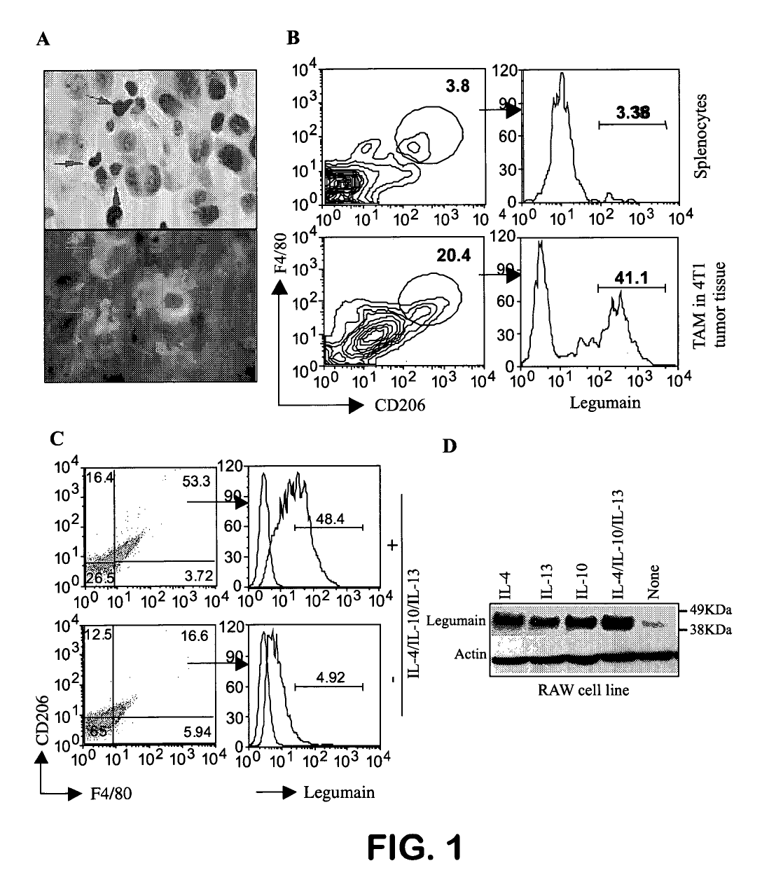

Legumain serves as a target to kill TAMs overexpressed during tumor progression. It is well known that TAMs play an important role in tumor progression and metastasis. Therefore, targeting of these M2 macrophages represents a novel anti-tumor strategy. Legumain was initially identified as a significant marker molecule of TAMs, because of its high level of expression on these cells in the tumor microenvironment and stroma. TAMs were isolated from murine 4T1 breast tumor tissue. Flow cytometry (FACS) demonstrated that legumain was highly overexpressed on CD206 and F4/80 double positive M2 macrophages, especially when compared to normal M1 macrophages in the spleen (FIG. 1, Panel B). This result was also confirmed by immunohistochemical analyses indicating that TAMs could be visualized by H/E staining, and legumain overexpression was further indicated by double staining with anti-legumain antibody (Ab) (green) combined with anti-CD68 Ab (red) (FIG. 1, Panel A). These data demonstrate that infiltrating TAMs are a disproportionally large cell subpopulation in 4T1 tumor tissue and that legumain is a potentially effective target for killing TAMs.

Induction of legumain expression on TAMs by Th2 cytokines. A murine macrophage cell line, RAW, co-cultured with these cytokines, was used to assess the extent to which legumain expression on TAMs was induced by Th2 cytokines, such as IL-4, IL-10 and IL-13. A significant increase in CD206⁺, F4/80⁺ expression by these RAW cells was observed, concurrent with an upregulation of legumain (FIG. 1, Panel C). These results were confirmed by Western blotting (FIG. 1, Panel D). No evidence for legumain expression by tumor cell lines was found when cultured with these same cytokines. These findings indicate that Th2 cytokines, such as IL-4, IL-10 and IL-13, are released by tumor and other tumor stromal cells and accumulate in the tumor microenvironment. In this environment, the cytokines can potentially induce the proliferation and transformation from M1 macrophages to a population with M2 phenotype, which overexpresses legumain.

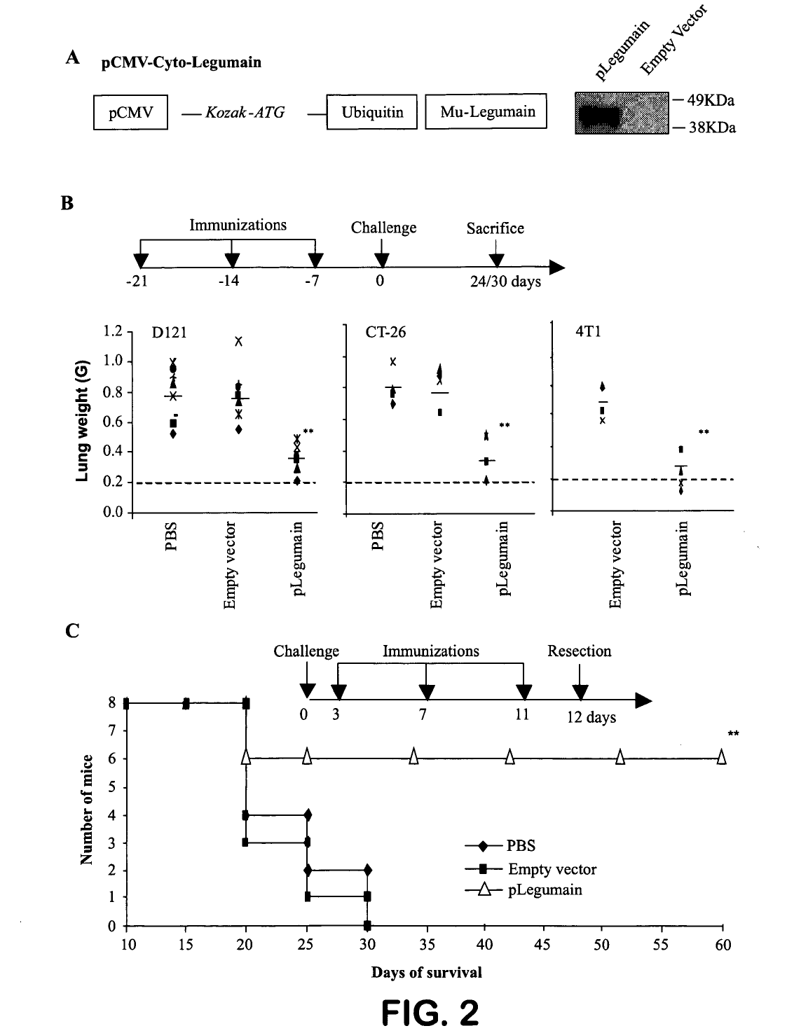

Targeting of TAMs suppresses tumor progression. Growth and metastases of tumors are highly coordinated with the presence of TAMs, and therefore targeting of this macrophage subpopulation leads to suppression in tumor growth and metastases. An expression vector for a DNA composition encoding legumain was prepared to induce an immune response against these TAMs. Attenuated Salmonella typhimurium bacteria (strain RE88) were transfected with a plasmid vector encoding legumain to provide a delivery vehicle for legumain DNA to immune cells. A control composition was prepared by incorporating an empty plasmid vector into the same strain of attenuated bacteria. FIG. 2, Panel A schematically depicts a vector construction map based on the pCMV/myc/cyto vector backbone, incorporating DNA encoding legumain. The gene encoding legumain was fused to the C-terminus of mutant polyubiquitin (pLegumain) to improve antigen processing in the proteasome. The entire fragment was then inserted between the PstI and NotI restriction sites of the plasmid. Legumain expression was demonstrated by Western blotting. In both prophylactic and therapeutic setting, reducing the number of TAMs using a legumain-based DNA composition of the present invention effectively inhibited spontaneous 4T1 breast cancer metastases and metastases of D121 non-small cell lung and CT26 colon carcinomas in mouse models.

In a prophylactic setting, C57BL/6J mice were immunized three times with either phosphate buffered saline (PBS; control group 1), attenuated S. typhimurium incorporating the empty vector (control group 2) or a DNA composition of the invention (pLegumain-transfected attenuated S. typhimurium; treatment group). One week after the last immunization, these mice were challenged intravenously (i.v.) with about 2×10⁵ D121 non-small lung tumor cells. About 24 days thereafter, lung metastases were measured and analyzed. In the two control groups, the average lung weight was significantly greater than that of the treatment group (FIG. 2, Panel B). Similar results were obtained in the CT26 colon tumor model and the 4T1 breast cancer model in syngeneic BALB/c mice (FIG. 2, Panel B).

In a more demanding therapeutic setting, BALB/c mice were first challenged with 4T1 breast cancer cells and then immunized three times with the pLegumain-based DNA composition (treatment group), PBS (control), or an empty vector composition (control), as described above. Twelve days after challenge with 4T1 tumor cells, the primary tumor was surgically excised. The life span curve for the control and treatment groups indicated that 75% (6/8) of the mice immunized with pLegumain survived for 3 months. In contrast, mice in the control groups all died within one month (FIG. 2, Panel C). These data indicate that the legumain-based DNA compositions of the present invention effectively suppress tumor cell growth and metastases in mouse models of 4T1 breast cancer, D121 non-small cell lung cancer and CT-26 colon carcinoma. Combined with surgery, this treatment could indeed significantly extend the life span of mice by inhibiting tumor cell metastases in these very challenging therapeutic mouse tumor models.

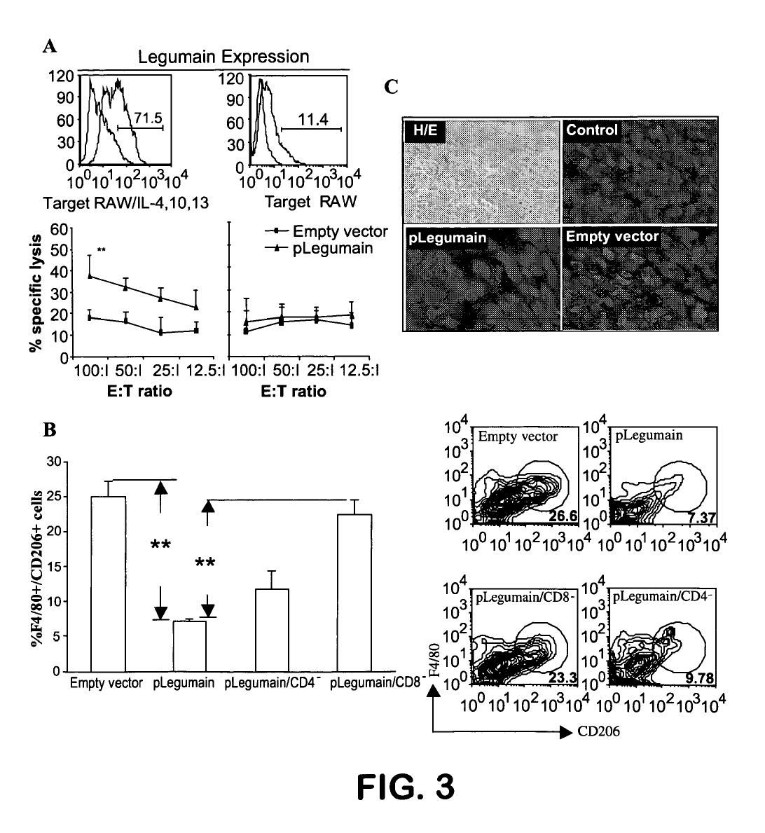

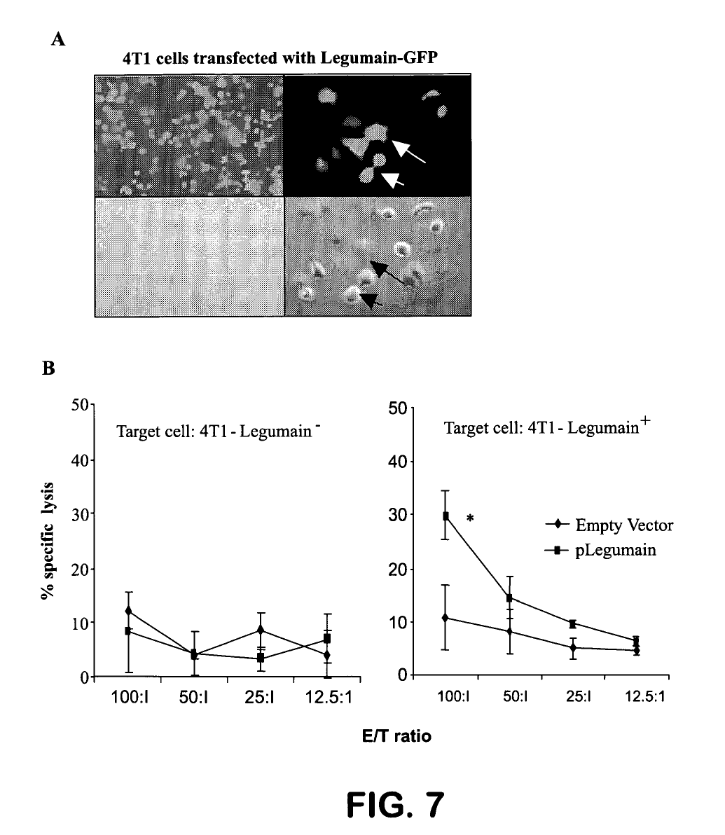

Targeting legumain induces a specific CD8+ CTL response decreasing TAM populations in the tumor stroma. Immunization against legumain induced a specific T cell response against TAMs that highly express this asparaginyl endopeptidase. The specific T cell response was demonstrated by a ⁵¹Cr release assay, where splenocytes isolated from mice successfully immunized with a DNA composition of the invention were effective in killing RAW macrophages, which expressed high levels of legumain after culture with cytokines IL-4, IL-10 and IL-13. These same splenocytes failed to induce cytotoxic killing of cells that did not express legumain (FIG. 3, Panel A), indicating a high degree of specificity for this T cell response against legumain. Additionally, the same result was obtained by using legumain transfected cells as targets in ⁵¹Cr release assays (FIG. 7). Furthermore, the results depicted in FIG. 3, Panel B demonstrate a dramatic decrease in the F4/80⁺/CD206⁺ macrophage population after treatment with a legumain-based DNA composition of the invention. These data were also confirmed by immunohistochemical staining, as shown in FIG. 3, Panel C.

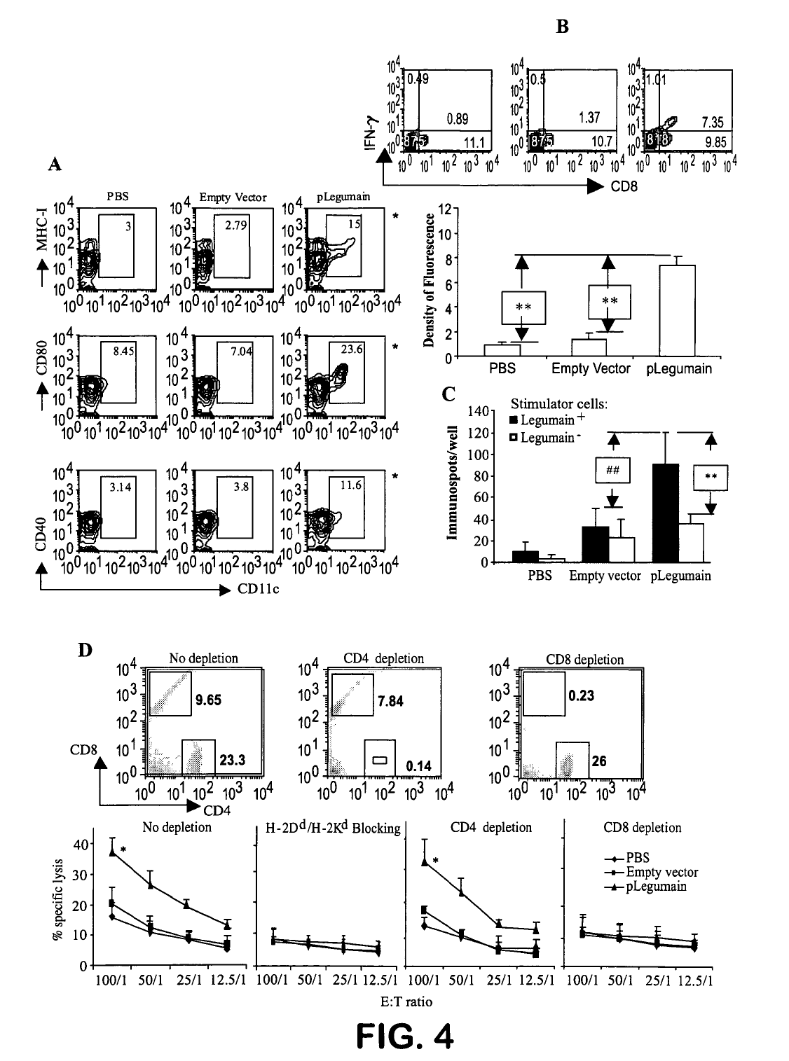

MHC-class I restricted CD8⁺ CTLs are specifically active against TAMs. While not wishing to be bound by theory, it is believed that the transfected bacteria are taken up by Peyer's patches in the gut, where the legumain DNA is then incorporated into immune cells, such as macrophages and dendritic cells (DCs), which then express the legumain DNA. DCs in Peyer's patches of successfully immunized mice were found to be activated 3 days after vaccination with pLegumain, as indicated by the upregulated DC activation markers, CD40, CD80, and MHC-I (FIG. 4, Panel A). CD8⁺ T cell activation was also found to be specific for legumain, as indicated by double staining for INF-gamma and CD8 on splenocytes obtained from successfully vaccinated mice, (FIG. 4, Panel B), and by the specific release of INF-gamma by activated T cells stimulated with legumain-positive cells (FIG. 4, Panel C). In addition, in vivo immune depletion of CD4⁺ or CD8⁺ T cells revealed that only CD8⁺ T cells play a major role in the specific cytotoxic killing of TAMs since only their depletion completely abrogated this killing effect. This specific cytotoxity was MHC-class I antigen restricted, since killing was specifically inhibited by anti-H-2Dd/H-2 Kd antibodies (FIG. 4, Panel D). Taken together, these results indicate that the legumain-based DNA compositions of the invention first activated DCs in Peyer's patches, after which these cells presented legumain peptides through the MHC-class I antigen pathway to the T cell receptor (TCR) on activated CD8⁺ T cells, resulting in a specific cytotoxic CD8⁺ T cell response abrogating TAMs.

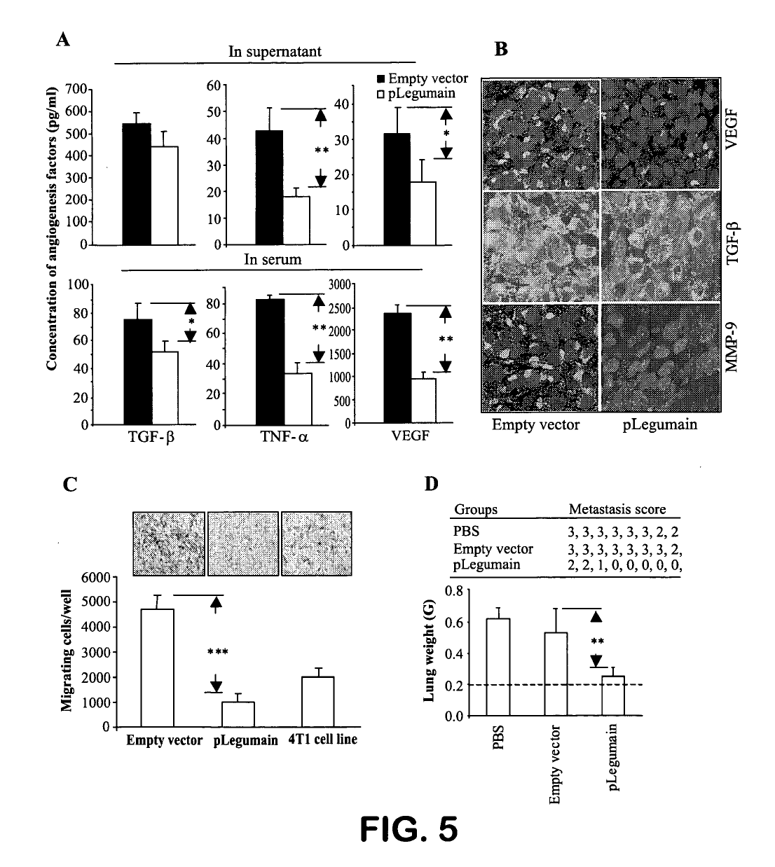

Abrogation of TAMs in the tumor stroma reduces the release of tumor growth factors and pro-angiogenesis factors, as well as inhibits tumor cell migration and metastases. TAMs can influence tumor metastasis in several ways, since they secrete a wide variety of tumor growth factors, pro-angiogenesis factors, and tumor-associated enzymes that stimulate tumor angiogenesis, as well as tumor growth and metastasis. In an effort to assess the extent to which the elimination of TAMs actually reduced the release of some of these factors, serum and tumor tissue cells were collected from vaccinated mice and from suitable control animals. Freshly isolated tumor cells were cultured, and their supernatants collected at 24 and 48 hours, respectively. An ELISA, performed to quantify TNF-alpha, VEGF and TGF-beta, indicated a significant reduction in TNF-alpha and VEGF in both tumor cell supernatants and serum. TGF-beta was reduced only in cell supernatants, but not in serum (FIG. 5, Panel A).

Immunohistological staining confirmed a decrease in the expression of these factors in tumor tissue (FIG. 5, Panel B). In addition, a significant decrease in tumor cell migration was observed when comparing treatment and control groups (FIG. 5, Panel C) in a migration and invasion assay. This result indicates that the characteristics of tumor cells changed after the vaccine-induced remodeling of the tumor microenvironment caused by the reduction in TAMs. The ability to form tumor metastases was confirmed in an in vivo assay by determining a metastasis score and lung weights 24 days after primary tumor excision in a therapeutic setting. The metastasis score and lung weights for treated mice decreased significantly when compared with the two control groups (FIG. 5, Panel D).

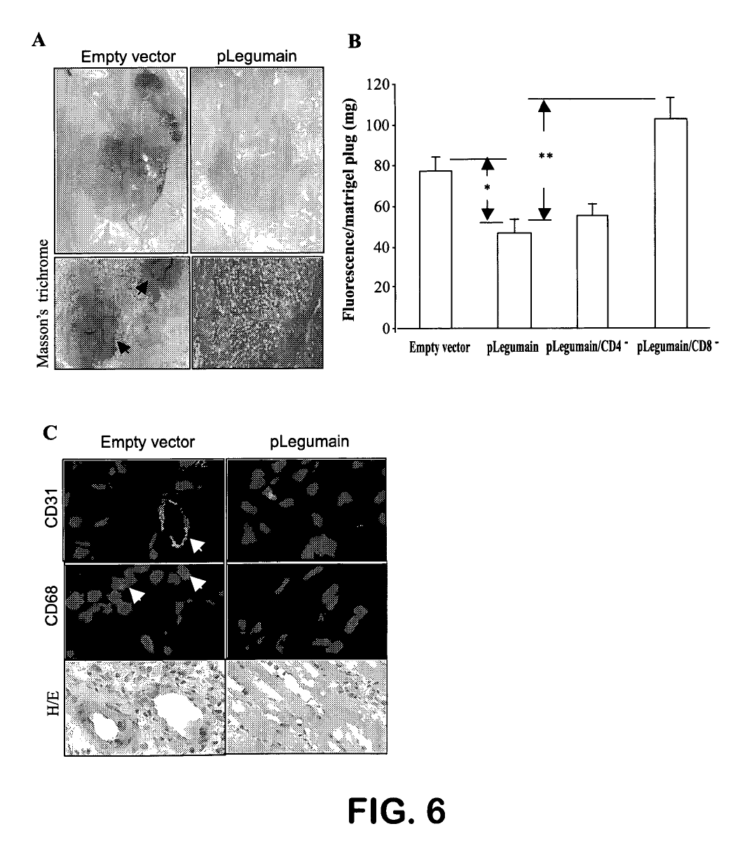

Elimination of TAMs in the tumor stroma results in reduction of tumor angiogenesis. A marked anti-angiogenic effect was observed after eliminating TAMs in the tumor stroma, which may be in part due to the fact that these M2 macrophages produce a wide range of pro-angiogenesis factors. Matrigel assays detected new blood vessel grown in vivo, which was quantified by staining the endothelium with FITC-labeled isolectin B4. These results clearly show that vessel growth was significantly reduced after vaccination with a DNA composition of the present invention (FIG. 6, Panel B). A much greater number of blood vessels were observed to be growing in Matrigel plugs in control mice immunized with empty vector compared to those treated with the legumain DNA composition, as determined by digital imaging and staining with Masson Trichrome (FIG. 6, Panel A). Furthermore, an immunochemical histology assay was performed to assess the type of cells that actually migrated into the Matrigel plugs. Confocal microscopic images indicated that endothelial cells expressing CD31 or macrophages expressing CD68, grew or migrated to a considerable extent into Matrigel plugs in the empty vector control group, but did so to a considerable lesser extent in the treatment group (FIG. 6, Panel C).

Transgenic legumain-expressing tumor cells are targeted by splenocytes from mice immunized with a DNA composition of the invention. In a model study, the 4T1 cell line was stably transfected by a retrovirus harboring the legumain plasmid and then used as target cells (FIG. 7, Panel A, left photos have a 5× magnification; right photos have a 35× magnification). Photomicrograph images were taken 2 days after transfection. The positive cells are indicated by arrows in FIG. 7, Panel A. A ⁵¹Cr release assay was performed; data are shown in FIG. 7, Panel B. Splenocytes isolated from mice immunized with the pLegumain DNA composition were effective in killing the 4T1 cells that were transfected with legumain (*P<0.01, compared to empty vector control groups). The tumor specific T cell-mediated killing was specific for legumain since 4T1 cells, lacking in legumain expression, were not lysed.

The present invention provides a new paradigm for tumor treatment, i.e., a reduction in the density of TAMs in the tumor stroma decreases the release of factors potentiating tumor growth and angiogenesis, which remodels the tumor micro-environment and markedly suppresses tumor cell proliferation, vascularization and metastasis. Targeting TAMs in the tumor stroma might raise the concern that abrogation of these cells could interfere with the normal immunological functions of these important components of the innate immune system. This is not the case, however.

Circulating monocytes are versatile precursors with the ability to differentiate into the various forms of specialized macrophages. In fact, the cytokine milieu profoundly affects the differentiation and function of tissue macrophages. Macrophages activated by bacterial products and Th1 cytokines are regarded as being of the M1 phenotype, i.e., classically activated macrophages with high bactericidal activity and cytotoxic function against tumor cells. In contrast, macrophages activated by Th2 cytokines, such as IL-4, and IL-13, or immunosuppressors, such as vitamin D3 and IL-10, are classified as having the M2 phenotype, which is characterized by a low cytotoxic function, but high tissue-remodeling activity.

M1 cells have immunostimulatory properties and defend the host against pathogenic infections, while M2 cells attenuate acute inflammatory reactions, potently scavenge cellular debris, and secrete a variety of pro-growth and angiogenesis factors essential for the repair of injured tissues. In addition, macrophages derived from healthy or inflamed tissue are capable of lysing tumor cells, expressing immunostimulatory cytokines, and presenting tumor-associated antigens to stimulate the proliferation and anti-tumor functions of T and NK cells. M2 macrophages such as TAMs, show reduced levels of these activities. This may be the result of their exposure to tumor-derived anti-inflammatory molecules such as IL-4, IL-10, TGF-beta1, and prostaglandin E2. Indeed, Mantovani and colleagues have suggested that exposure to IL-4 and IL-10 may induce monocytes in tumors to develop into polarized type II or M2 macrophages (Mantovani et al. 2002, Trends Immunol. 23:549-555).

To the extent that they have been previously investigated, differentiated mature TAMs have a phenotype and function similar to Type II macrophages (Mantovani et al. 2004, Eur. J. Cancer 40:1660-1667). Therefore, cytokines present in the tumor microenvironment have the potential to promote and orient the differentiation of recruited mononuclear phagocytes. Indeed, a growing body of evidence indicates that TAMs are skewed toward M2 macrophages in the tumor microenvironment, and produce a variety of pro-tumor growth and angiogenesis factors, as well as immunosuppressive molecules. Thus, the presence of TAMs at the tumor site and the continuous expression and release of their products may favor, rather than antagonize tumor progression and metastasis.

TAMs express abundant levels of CD206, a mannose receptor that is up-regulated on M2 macrophages following exposure to IL-4 and IL-13 (Porcheray et al. 2005, Clin. Exp. Immunol. 142:481-489). As demonstrated herein, this population of macrophages expresses a high level of legumain. Importantly, Th2 cytokines IL-4, IL-10 and IL-13 up-regulate the expression of CD206 and legumain on the macrophage cell line RAW. This finding can best be understood when one considers that macrophages are derived from peripheral blood, and differentiate into M2 macrophages once they are recruited into tumor sites where IL-4, IL-13 and IL-10 are released by tumor cells and tumor stromal cells (Stein et al. 1992, J. Exp. Med. 176:287-292). Thus, targeting of M2 macrophages expressing legumain not only benefits suppression of tumor growth and metastases, but also maintains the normal functions of macrophages with M1 phenotype.

The relationship between infiltration by TAMs and prognosis in tumor patients has also been indicated by several studies, which concluded that the greater the macrophage infiltration, the worse the prognosis (see e.g., Wyckoff et al. 2004, Cancer Res. 64:7022-7029). Several lines of evidence indicate that a symbiotic relationship exists in the tumor stroma between cancer cells and TAMs, whereby cancer cells attract TAMs and sustain their survival, while TAMs respond to tumor-derived molecules by producing important growth factors and extracellular matrix enzymes. This, in turn, stimulates tumor proliferation, angiogenesis, and invasion of surrounding tissues (see e.g., Wyckoff et al. 2004, Cancer Res. 64:7022-7029). Thus, the attenuation of TAMs in the tumor environment provides an effective strategy to remodel the tumor stroma and to alter the tumor microenvironment.

DNA compositions of the present invention evoked a robust CTL response against this legumain, which is an asparaginyl endopeptidase that functions as a stress protein, and is highly overexpressed by TAMs. The anti-legumain immune response was shown to be MHC-I class I antigen-restricted and CD8⁺ T cell specific. Importantly, the present invention demonstrates that after immunization with the legumain-based DNA composition of the invention, the density of double positive CD206⁺ and F4/80⁺ macrophages, i.e. TAMs, decreased dramatically. In addition, a variety of factors such as VEGF, MMP-9 and TGF-beta that are released by TAMs were shown to be present at low levels in both the supernatant of cultured tumor cells and mouse serum. It is well known that VEGF and metalloproteinase MMP-9 play important roles during the formation of the tumor vasculature and initiation of tumor angiogenesis. TAMs are significant in this regard, since they produce both VEGF and MMP-9. Progressively intensifying angiogenesis is associated with the upregulated expression of VEGF and extracellular proteases, such as MMP-9, whereas TGF-beta is known to be an important growth factor involved in the migration of tumor cells towards blood vessels. In fact, TGF-beta can provide proliferative and anti-apoptotic signals to tumor cells as well as activate urokinase-type plasminogen activators (uPA) that might contribute to the extracellular matrix breakdown which is required for vascular invasion to occur.

Significantly, the present invention demonstrates that once TAMs have been abrogated in the tumor tissue by specific CD8⁺ cytotoxic T lymphocytes (CTLs), the tumor cells changed their character by becoming less malignant and less invasive. The formation of a neovasculature in tumor tissues also was drastically reduced by treatment with a DNA composition of the present invention. Additionally, TAMs reportedly are involved in immune suppression and tolerance in the tumor microenvironment, and may inhibit T cell responses by inducing apoptosis of activated T cells via up-regulation of NO, PGs, TNF-alpha release and arginase activity (see e.g., Saio et al. 2001, J. Immunol. 167:5583-5593). After abrogation of TAMs, specific CD8⁺ T cell activity was markedly up-regulated, further indicating that the anti-TAM approach of the present invention provides an effective strategy to break immune tolerance against tumors.

In the 4T1 spontaneous mouse breast carcinoma metastasis model, a surprisingly significant increase in life span was obtained, in which 75% (6 out of 8) mice survived up to 3 months after 4T1 tumor cell inoculation into the mammary gland, once surgical resection of the primary tumor was followed by treatment with the legumain-based DNA composition of the invention. Even more unexpectedly, 62% (5 out of 8) mice revealed no lung metastases at all. Similar results were obtained in prophylactic settings in the other two tumor models, i.e. D121-non-small cell lung carcinoma and CT-26 colon carcinoma. These additional confirmatory data demonstrate that targeting of TAMs to remodel the tumor microenvironment is a potentially universal anti-tumor strategy, which suppresses tumor cell invasion and metastases by reducing the concentration of factors released by TAMs that otherwise promote tumor growth and metastasis.

In Example 2, a mammalian expression vector with an ER signal (pCMV/Myc/ER) was utilized for construction of a minigene DNA composition of the present invention. The ER signal peptide directs the protein into the secretory compartment. The vector also included a C-terminal peptide that retains the protein in the endoplasmic reticulum (ER). The peptides encoded by the DNA composition were processed in the ER of immune cells in the gut and then bound to MHC class I antigen binding sites to be finally presented to T cell receptors, which induced a legumain-specific T cell response against TAMs in the mice which express legumain. These constructs create effective minigene vaccines which can induce an effective CD8⁺ T cell response against tumors of different organs in mouse tumor models.

Additional experiments with D121 non-small cell carcinoma cells in syngeneic C57/BL mice showed that both the pCMV-Db/Dd and pCMV-Kb/Kd minigene constructs provided effective anti-TAM immune response. In contrast, the results with D2F2 cells in BALB/c mice indicates that the pCMV-Kb/Kd construct was considerably more effective than either the pCMV-Db/Dd or the pCMV-Kb/Kd construct. A possible explanation for this finding is that there is a much greater number of legumain peptides predicted to bind to H-2Dd/Kd with higher affinity than those binding to H-2Dd/Kb molecules. According to the BioInformatics & Molecular Analysis Section (BIMAS) of NIH, the predictive scores of legumain peptides binding to H-2Dd and H-2 Kd are considerably higher than the corresponding scores for H-2 Db and H-2 Kb, whereas the binding score of H-2 Kd is the highest (FIG. 20).

Considering that the T cell receptor repertoire is almost unlimited, there may be a much higher number of peptide-H-2Dd/Kd complexes that can be recognized by the CTLs in the BALB/c mice. Moreover, the H-2D peptide had a much higher antigenicity index than two of three H-2K peptides (FIG. 20). Based on these data, antigenicity is less predictive than the MHC binding score with respect to the anti-TAM immune response. The legumain-based minigene compositions of the present invention induced an effective protection against tumors by attacking TAMs in breast tumor models. The tumor protection induced by the Kd-based minigene vaccines led to an attack on TAMs in the D2F2 breast carcinoma microenvironment in syngeneic in BALB/c mice. This protective immune response was found to be mediated by CD8⁺ T cells, which specifically kill legumain⁺ TAMs, and also results in a marked suppression of tumor angiogenesis. Importantly, the minigene DNA constructs of the invention proved to be of similar efficacy as a vaccine encoding the whole legumain gene. Similarly, both Dd and Kd-based constructs provided anti-TAM responses in a D121 cancer model in C57/BL mice. Taken together, these data represent the first anti-legumain minigene composition effective against TAMs and that this strategy can be particularly useful for individuals with different genetic background, and thereby provide a simple, more safe and flexible alternative to the whole-gene vaccine strategy in breast cancer treatment.

Numerous variations and modifications of the embodiments described above can be effected without departing from the spirit and scope of the novel features of the invention. No limitations with respect to the specific embodiments illustrated herein are intended or should be inferred.

1. A DNA composition comprising a DNA minigene construct that encodes for a polypeptide comprising a plurality of immunogenic fragments of a cysteine endopeptidase that is expressed in tumor-associated cells, the plurality of immunogenic fragments being joined together serially by a linker peptide between each successive fragment in the polypeptide, wherein the polypeptide is capable of eliciting an immune response against the tumor-associated cells, is expressible in immune cells, and is incorporated in a pharmaceutically acceptable carrier.

2. The DNA composition of claim 1 wherein the cysteine endopeptidase is legumain.

3. The DNA composition of claim 1 wherein the DNA minigene construct encodes for immunogenic fragments of human legumain (SEQ ID NO: 2).

4. The DNA composition of claim 1 wherein the linker peptide between each successive immunogenic fragment comprises at least three amino acid residues.

5-8. (canceled)

9. The DNA composition of claim 1 wherein the DNA construct is incorporated in an attenuated AroA⁻, dam⁻ Salmonella typhimurium bacterial vector.

10. The DNA composition of claim 1 wherein the DNA minigene construct comprises portions of SEQ ID NO: 1 that encode immunogenic fragments of human legumain.

11. The DNA composition of claim 1 which further comprises a DNA construct encoding an immune effector protein expressible in immune cells.

12. The DNA composition of claim 11 wherein the immune effector protein is a cytokine.

13. The DNA composition of claim 12 wherein the cytokine is CCL21, IL-2, or CD40LT.

14. The DNA composition of claim 1 wherein the DNA minigene construct further encodes an endoplasmic reticulum leader peptide sequence linked to the N-terminus of the polypeptide.

15. A method of inhibiting tumor growth or tumor metastases in a mammal comprising administering to a mammal a DNA composition of claim 1 comprising a DNA minigene construct that encodes for a plurality of immunogenic fragments of a cysteine endopeptidase that is expressed in tumor-associated cells; the composition being administered in an amount sufficient to elicit an immune response against the tumor-associated cells.

16. The method of claim 15 wherein the tumor associated cells are tumor-associated macrophages.

17-18. (canceled)

19. A method of inhibiting tumor growth or tumor metastases in a mammal comprising the steps of:
administering to a mammal a DNA composition of claim 1 comprising a DNA minigene construct encoding a plurality of immunogenic fragments of a cysteine endopeptidase that is expressed in tumor-associated cells; and
subsequently administering to the mammal an antitumor effective amount of an antitumor chemotherapeutic agent; the DNA composition being administered in an amount sufficient to elicit an immune response against tumor-associated macrophages in the mammal that express the cysteine endopeptidase.

20. (canceled)

21. The method of claim 19 wherein the chemotherapeutic agent is doxorubicin.

22. (canceled)

23. A method of inhibiting tumor-associated macrophage production in a mammal benefiting from said inhibition, which comprises administering to a mammal an immune response eliciting amount of a DNA composition of claim 1 comprising a DNA minigene construct that encodes for a plurality of immunogenic fragments of a cysteine endopeptidase that is expressed in the tumor-associated macrophages.

24-25. (canceled)

26. An article of manufacture comprising a DNA composition of claim 1 packaged in a hermetically sealed, sterile container, the container having a label affixed thereto, the label bearing printed material identifying the composition and providing information useful to an individual administering said composition to a patient.

27. A plasmid vector comprising a DNA minigene construct that encodes for a polypeptide comprising a plurality of immunogenic fragments of legumain joined together serially by a linker peptide between each successive fragment in the polypeptide, and which is capable of being expressed in immune cells and eliciting an immune response against tumor-associated cells that overexpress legumain.

28. The vector of claim 27 wherein the legumain is human legumain having the amino acid residue sequence consisting of SEQ ID NO: 2 or a protein that has at least 80% sequence identity therewith.

29. The vector of claim 27 further comprising a DNA construct encoding an immune effector protein.

30. The vector of claim 29 wherein the immune effector protein is a cytokine.

31. The vector of claim 30 wherein the cytokine is CCL21, IL-2, or CD40LT.

32. The vector of claim 27 wherein the linker peptide comprises at least three amino acid residues.

33. The vector of claim 32 wherein each linker peptide comprises the amino acid residues sequence AAA or AAY.

34. A DNA composition comprising a DNA construct that encodes for a cysteine endopeptidase that is expressed in tumor-associated cells, or at least one fragment thereof capable of eliciting an immune response against the tumor-associated cells, which is expressible in immune cells, and is incorporated in a pharmaceutically acceptable carrier.

35. The DNA composition of claim 34 wherein the cysteine endopeptidase is human legumain.


SEQUENCE LISTING (21 sequences)

SEQ ID NO: 1 - DNA, 1302 bp, Homo sapiens
atggtttggaaagtagctgtattcctcagtgtggccctgggcattggtgccgttcctatagatgatcctgaagatggaggcaagcactgggtggtgatcgtggcaggttcaaatggctggtataattataggcaccaggcagacgcgtgccatgcctaccagatcattcaccgcaatgggattcctgacgaacagatcgttgtgatgatgtacgatgacattgcttactctgaagacaatcccactccaggaattgtgatcaacaggcccaatggcacaggtgtctatcagggagtcccgaaggactacactggagaggatgttaccccacaaaatttccttgctgtgttgagaggcgatgcagaagcagtgaagggcataggatccggcaaagtcctgaagagtggcccccaggatcacgtgttcatttacttcactgaccatggatctactggaatactggtttttcccaatgaagatcttcatgtaaaggacctgaatgagaccatccattacatgtacaaacacaaaatgtaccgaaagatggtgttctacattgaagcctgtgagtctgggtccatgatgaaccacctgccggataacatcaatgtttatgcaactactgctgccaaccccagagagtcgtcctacgcctgttactatgatgagaagaggtccacgtacctgggggactggtacagcgtcaactggatggaagactcggacgtggaagatctgactaaagagaccctgcacaagcagtaccacctggtaaaatcgcacaccaacaccagccacgtcatgcagtatggaaacaaaacaatctccaccatgaaagtgatgcagtttcagggtatgaaacgcaaagccagttctcccgtccccctacctccagtcacacaccttgacctcacccccagccctgatgtgcctctcaccatcatgaaaaggaaactgatgaacaccaatgatctggaggagtccaggcagctcacggaggagatccagcggcatctggatgccaggcacctcattgagaagtcagtgcgtaagatcgtctccttgctggcagcgtccgaggctgaggtggagcagctcctgtccgagagagccccgctcacggggcacagctgctacccagaggccctgctgcacttccggacccactgcttcaactggcactcccccacgtacgagtatgcgttgagacatttgtacgtgctggtcaacctttgtgagaagccgtatccacttcacaggataaaattgtccatggaccacgtgtgccttggtcactattaa

SEQ ID NO: 2 - protein, 433 aa, Homo sapiens [sequence omitted]

SEQ ID NO: 3 - DNA, 1308 bp, Mus musculus
atgacctggagagtggctgtgcttctcagcctggtgctgggtgctggtgccgttcccgtcggtgtggacgatcccgaggatggaggcaagcactgggtggtgattgtggcgggctccaatggctggtataattaccgacaccaggcagacgcatgccacgcctaccagatcatccaccggaacgggattcctgacgagcagatcatagtgatgatgtatgacgacattgccaactctgaagaaaaccctaccccaggtgttgtgatcaaccgacctaacggcacagatgtatacaagggagtcctgaaggactacaccggagaggatgtgactccagagaatttcctcgccgtgctgagaggtgacgcagaagctgtgaagggcaaagggtctggaaaagtcttgaagagtggcccccgagatcatgtcttcatttacttcaccgaccacggagccaccgggatcctggtgtttcctaatgatgatcttcatgtcaaggacctgaataagactattcgctacatgtatgaacacaaaatgtaccagaagatggtgttctacattgaagcttgtgagtctggctccatgatgaaccacctgcccgacgacatcaacgtttatgcaactactgcggccaaccccaaggagtcatcttatgcctgctactacgacgaggagaggggcacttacctgggtgactggtacagcgtcaactggatggaagactccgatgtggaggacctgaccaaagagacccttcacaagcagtaccacctggtcaagtcccacaccaacaccagccatgtcatgcaatatgggaacaaatctatctctaccatgaaagtgatgcagtttcagggaatgaagcacagagccagttcccccatctccctgcctccggtcacacaccttgacctcacccccagccctgacgtgcccctgaccatcttgaagaggaagctgctgagaaccaacgacgtgaaggaatcccagaatctcattgggcagatccagcaatttctggatgccaggcacgtcattgagaagtctgtgcacaagatcgtttccctgctggcgggatttggggaaactgctgagagacatctgtcagagaggaccatgctcacagcacatgactgctaccaggaggctgtaacccacttccgcacacactgctttaactggcactctgtcacgtacgagcatgccttgcggtacttgtatgtgctggccaatctctgtgaggcaccatatccgattgacaggatagagatggccatggacaaagtgtgtcttagtcactactga

SEQ ID NO: 4 - protein, 435 aa, Mus musculus
MTWRVAVLLSLVLGAGAVPVGVDDPEDGGKHWVVIVAGSNGWYNYRHQADACHAYQIIHRNGIPDEQIIVMMYDDIANSEENPTPGVVINRPNGTDVYKGVLKDYTGEDVTPENFLAVLRGDAEAVKGKGSGKVLKSGPRDHVFIYFTDHGATGILVFPNDDLHVKDLNKTIRYMYEHKMYQKMVFYIEACESGSMMNHLPDDINVYATTAANPKESSYACYYDEERGTYLGDWYSVNWMEDSDVEDLTKETLHKQYHLVKSHTNTSHVMQYGNKSISTMKVMQFQGMKHRASSPISLPPVTHLDLTPSPDVPLTILKRKLLRTNDVKESQNLIGQIQQFLDARHVIEKSVHKIVSLLAGFGETAERHLSERTMLTAHDCYQEAVTHFRTHCFNWHSVTYEHALRYLYVLANLCEAPYPIDRIEMAMDKVCLSHY

SEQ ID NO: 5 - DNA, 814 bp, Homo sapiens
atcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacgtatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 6 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - DNA, 852 bp, Homo sapiens
cttgcagctgcccacctcaccctcagctctggcctcttactcaccctctaccacagacatggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatagcccagtgagcagcctggagccctggagaccccaccagcctcaccaacgcttgaagcctgaacccaagatgcaagaaggaggctatgctcaggggccctggagcagccaccccatgctggccttgccacactctttctcctgctttaaccaccccatctgcattcccagctctaccctgcatggctgagctgcccacagcaggccaggtccagagagaccgaggagggagagtctcccagggagcatgagaggaggcagcaggactgtccccttgaaggagaatcatcaggaccctggacctgatacggctccccagtacaccccacctcttccttgtaaatatgatttatacctaactgaataaaaagctgttctgtcttcccacccgc

SEQ ID NO: 8 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 9 - DNA, 1816 bp, Homo sapiens
cttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaacccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaaaa

SEQ ID NO: 10 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 11 - DNA, 1533 bp, Artificial Sequence
  Synthetic ubiquinated murine legumain
atgcagatcttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggcacctggagagtggctgtgcttctcagcctggtgctgggtgctggtgccgttcccgtcggtgtggacgatcccgaggatggaggcaagcactgggtggtgattgtggcgggctccaatggctggtataattaccgacaccaggcagacgcatgccacgcctaccagatcatccaccggaacgggattcctgacgagcagatcatagtgatgatgtatgacgacattgccaactctgaagaaaaccctaccccaggtgttgtgatcaaccgacctaacggcacagatgtatacaagggagtcctgaaggactacaccggagaggatgtgactccagagaatttcctcgccgtgctgagaggtgacgcagaagctgtgaagggcaaagggtctggaaaagtcttgaagagtggcccccgagatcatgtcttcatttacttcaccgaccacggagccaccgggatcctggtgtttcctaatgatgatcttcatgtcaaggacctgaataagactattcgctacatgtatgaacacaaaatgtaccagaagatggtgttctacattgaagcttgtgagtctggctccatgatgaaccacctgcccgacgacatcaacgtttatgcaactactgcggccaaccccaaggagtcatcttatgcctgctactacgacgaggagaggggcacttacctgggtgactggtacagcgtcaactggatggaagactccgatgtggaggacctgaccaaagagacccttcacaagcagtaccacctggtcaagtcccacaccaacaccagccatgtcatgcaatatgggaacaaatctatctctaccatgaaagtgatgcagtttcagggaatgaagcacagagccagttcccccatctccctgcctccggtcacacaccttgacctcacccccagccctgacgtgcccctgaccatcttgaagaggaagctgctgagaaccaacgacgtgaaggaatcccagaatctcattgggcagatccagcaatttctggatgccaggcacgtcattgagaagtctgtgcacaagatcgtttccctgctggcgggatttggggaaactgctgagagacatctgtcagagaggaccatgctcacagcacatgactgctaccaggaggctgtaacccacttccgcacacactgctttaactggcactctgtcacgtacgagcatgccttgcggtacttgtatgtgctggccaatctctgtgaggcaccatatccgattgacaggatagagatggccatggacaaagtgtgtcttagtcactactga

SEQ ID NO: 12 - protein, 510 aa, Artificial Sequence
  Synthetic ubiquinated murine legumain
MQIFVKTLTGKTITLEVEPSDTIENVKAKIQDKEGIPPDQQRLIFAGKQLEDGRTLSDYNIQKESTLHLVLRLRGGTWRVAVLLSLVLGAGAVPVGVDDPEDGGKHWVVIVAGSNGWYNYRHQADACHAYQIIHRNGIPDEQIIVMMYDDIANSEENPTPGVVINRPNGTDVYKGVLKDYTGEDVTPENFLAVLRGDAEAVKGKGSGKVLKSGPRDHVFIYFTDHGATGILVFPNDDLHVKDLNKTIRYMYEHKMYQKMVFYIEACESGSMMNHLPDDINVYATTAANPKESSYACYYDEERGTYLGDWYSVNWMEDSDVEDLTKETLHKQYHLVKSHTNTSHVMQYGNKSISTMKVMQFQGMKHRASSPISLPPVTHLDLTPSPDVPLTILKRKLLRTNDVKESQNLIGQIQQFLDARHVIEKSVHKIVSLLAGFGETAERHLSERTMLTAHDCYQEAVTHFRTHCFNWHSVTYEHALRYLYVLANLCEAPYPIDRIEMAMDKVCLSHY

SEQ ID NO: 13 - protein, 9 aa, Mus musculus
SGPRDHVFI

SEQ ID NO: 14 - protein, 9 aa, Mus musculus
LPPVTHLDL

SEQ ID NO: 15 - protein, 9 aa, Mus musculus
YDEERGTYL

SEQ ID NO: 16 - protein, 9 aa, Mus musculus
RYLYVLANL

SEQ ID NO: 17 - protein, 9 aa, Mus musculus
MYQKMVFYI

SEQ ID NO: 18 - protein, 9 aa, Mus musculus
TYLGDWYSV

SEQ ID NO: 19 - protein, 18 aa, Mus musculus
MGWSCIILFLVATATAHS

SEQ ID NO: 20 - protein, 10 aa, Mus musculus
EQKLISEEDL

SEQ ID NO: 21 - protein, 6 aa, Mus musculus
SEKDEL